(19) United States
(12) Patent Application Publication
(10) Pub. No.: US 2024/0043559 A1
(43) Pub. Date: Feb. 8, 2024

(54) ANTI-GPC3 ANTIBODIES AND METHODS OF USE

(71) Applicant: SHANGHAI HENLIUS BIOTECH, INC., Shanghai (CN)

(72) Inventors: Hassan ISSAFRAS, Fremont, CA (US); Wenfeng XU, Belmont, CA (US); Wei-Dong JIANG, Los Gatos, CA (US); Heungnam KIM, Pleasant Hill, CA (US)

(21) Appl. No.: 18/491,313

(22) Filed: Oct. 20, 2023

Related U.S. Application Data
(63) Continuation of application No. PCT/CN2022/088432, filed on Apr. 22, 2022.

(30) Foreign Application Priority Data
Apr. 23, 2021 (WO) PCT/CN2021/089233

(51) Int. Cl. C07K 16/30; A61K 39/00; A61P 35/00

(52) U.S. Cl. CPC C07K 16/303; A61K 39/4631; A61P 35/00; C07K 2317/569; C07K 2317/565; C07K 2317/52; A61K 2239/13

(57) ABSTRACT

Provided are antibodies and antibody derivatives that bind to GPC3 and methods of using the same. The antibody or antibody derivative comprises a single domain antibody that binds to GPC3.

This application claims the priority of International Patent Application No. PCT/CN2021/089233, filed on Apr. 23, 2021.

### FIELD

The present disclosure relates to antibodies and antibody derivatives that bind to GPC3 and methods of using the same.

### BACKGROUND

Glypican 3 (GPC3) is a GPI-linked heparan sulfate proteoglycan and cell surface oncofetal protein that is highly expressed in over 70% of hepatocellular carcinoma (HCC) biopsies. Patients with GPC3-positive HCC have a significantly lower disease-free survival rate than patients with GPC3-negative HCC. GPC3 is also present as soluble GPC3 (sGPC3) in peripheral blood of HCC patients, but not in the liver tissues of either healthy adults, pathological samples of fatty liver, or liver with cirrhosis, hepatitis, or injury, suggesting that GPC3 is a more reliable tumor marker than alpha-fetoprotein (AFP). GPC3 is also expressed on a variety of pediatric cancers such as hepatocellular carcinoma, majority of pediatric hepatoblastomas, Wilms tumors, rhabdoid tumors, certain germ cell tumor subtypes, and a minority of rhabdomyosarcomas. Additionally, mutations in the GPC3 gene lead to Simpson-Golabi-Behmel Syndrome, an X-linked overgrowth condition with a predisposition to GPC3-expressing cancers including hepatoblastoma and Wilms tumor. Accordingly, there is a need in the art for the development of therapeutic molecules and methods to target GPC3 for cancer treatment.

### SUMMARY OF THE INVENTION

The present disclosure provides isolated monoclonal antibodies and antibody derivatives that bind specifically to GPC3 with high affinity, including monospecific anti-GPC3 antibodies and multispecific antibodies that binds to GPC3 and one or more additional target. In certain embodiments, an antibody or antibody derivative disclosed herein comprises a single domain antibody that binds to GPC3. This disclosure further provides methods of making and using antibodies and antibody derivatives disclosed herein and pharmaceutical compositions comprising the same, e.g., for treating diseases and disorders, e.g., cancer. The invention is based, in part, on the discovery of novel single domain antibodies that bind to GPC3, which can target a tumor cell and/or increase an immune response against a tumor cell and thereby provide improved anti-tumor efficacy.

The present disclosure provides an antibody that binds to GPC3, comprising a single domain antibody that binds to GPC3. In certain embodiments, the single domain antibody binds to GPC3 with a KD of 1×10⁻⁷ M or less. In certain embodiments, the single domain antibody binds to GPC3 with a KD of 5×10⁻⁸ M or less. In certain embodiments, the single domain antibody binds to GPC3 with a KD of 1×10⁻⁸ M or less. In certain embodiments, the single domain antibody binds to GPC3 with a KD of between about 1×10⁻¹⁰ M and about 5×10⁻⁸ M. In certain embodiments, the single domain antibody comprises a VHH. In certain embodiments, the single domain antibody or the VHH comprises a heavy chain variable region (VH).

In certain embodiments, the single domain antibody cross-competes for binding to GPC3 with a reference anti-GPC3 single domain antibody comprising a heavy chain variable region comprising: a heavy chain variable region CDR1 comprising the amino acid sequence set forth in SEQ ID NO: 1, a heavy chain variable region CDR2 comprising the amino acid sequence set forth in SEQ ID NO: 2, and a heavy chain variable region CDR3 comprising the amino acid sequence set forth in SEQ ID NO: 3, or a heavy chain variable region CDR1 comprising the amino acid sequence set forth in SEQ ID NO: 5, a heavy chain variable region CDR2 comprising the amino acid sequence set forth in SEQ ID NO: 6, and a heavy chain variable region CDR3 comprising the amino acid sequence set forth in SEQ ID NO: 7.

In certain embodiments, the single domain antibody comprises a heavy chain variable region comprising: a) a heavy chain variable region CDR1 comprising an amino acid sequence of any one of SEQ ID NOs: 1 and 5, or a variant thereof comprising up to about 3 amino acid substitutions; b) a heavy chain variable region CDR2 comprising an amino acid sequence of any one of SEQ ID NOs: 2 and 6, or a variant thereof comprising up to about 3 amino acid substitutions; and c) a heavy chain variable region CDR3 comprising an amino acid sequence of any one of SEQ ID NOs: 3 and 7, or a variant thereof comprising up to about 3 amino acid substitutions.

In certain embodiments, the single domain antibody comprises a heavy chain variable region that comprises a CDR1 domain, a CDR2 domain and a CDR3 domain, wherein the CDR1 domain, the CDR2 domain and the CDR3 domain respectively comprise a CDR1 domain, a CDR2 domain and a CDR3 domain comprised in a reference heavy chain variable region comprising the amino acid sequence selected from the group consisting of SEQ ID NOs: 4, 8 and 12. In certain embodiments, the single domain antibody comprises a heavy chain variable region CDR1 comprising the amino acid sequence set forth in SEQ ID NO: 1, a heavy chain variable region CDR2 comprising the amino acid sequence set forth in SEQ ID NO: 2, and a heavy chain variable region CDR3 comprising the amino acid sequence set forth in SEQ ID NO: 3. In certain embodiments, the single domain antibody comprises a heavy chain variable region CDR1 comprising the amino acid sequence set forth in SEQ ID NO: 5, a heavy chain variable region CDR2 comprising the amino acid sequence set forth in SEQ ID NO: 6, and a heavy chain variable region CDR3 comprising the amino acid sequence set forth in SEQ ID NO: 7. In certain embodiments, the single domain antibody comprises a heavy chain variable region comprising an amino acid sequence having at least about 90% sequence identity to an amino acid sequence selected from the group consisting of SEQ ID NOs: 4, 8 and 12. In certain embodiments, the single domain antibody comprises a heavy chain variable region comprising the amino acid sequence set forth in SEQ ID NO: 4. In certain embodiments, the single domain antibody comprises a heavy chain variable region comprising the amino acid sequence set forth in SEQ ID NO: 8. In certain embodiments, the single domain antibody comprises a heavy chain variable region comprising the amino acid sequence set forth in SEQ ID NO: 12. In certain embodiments, the single domain antibody comprises a humanized framework.

In certain embodiments, the antibody comprises a Fc region. In certain embodiments, the Fc region comprises a human Fc region. In certain embodiments, the Fc region comprises a Fc region selected from the group consisting of the Fc regions of IgG, IgA, IgD, IgE and IgM. In certain embodiments, the Fc region comprises a Fc region selected from the group consisting of the Fc regions of IgG1, IgG2, IgG3 and IgG4. In certain embodiments, the Fc region comprises an IgG1 Fc region. In certain embodiments, the IgG1 Fc region comprising one or more mutation that enhances an antibody-dependent cell-mediated cytotoxicity (ADCC). In certain embodiments, the IgG1 Fc region comprises the mutations of L235V, F243L, R292P and Y300L, the mutations of S239D, A330L and I332E, or the mutations of L235V, F243L, R292P, Y300L and P396L. In certain embodiments, the IgG1 Fc region comprises the mutations of L235V, F243L, R292P and Y300L. In certain embodiments, the IgG1 Fc region comprises the mutations of S239D, A330L and I332E. In certain embodiments, the IgG1 Fc region comprises the mutations of L235V, F243L, R292P, Y300L and P396L.

In certain embodiments, the heavy chain variable region is linked to a Fc region via a linker. In certain embodiments, the linker is a peptide linker. In certain embodiments, the peptide linker comprises about four to about thirty amino acids. In certain embodiments, the peptide linker comprise an amino acid sequence selected from the group consisting of SEQ ID NOs: 16-50.

In certain embodiments, the antibody comprises a full-length immunoglobulin, a single-chain Fv (scFv) fragment, a Fab fragment, a Fab′ fragment, a F(ab′)2, an Fv fragment, a disulfide stabilized Fv fragment (dsFv), a (dsFv)2, a VHH, a VHH-Fc fusion, a Fv-Fc fusion, a scFv-Fc fusion, a scFv-Fv fusion, a diabody, a tribody, a tetrabody or any combination thereof.

In certain embodiments, the antibody is comprised in a multispecific antibody, e.g., a bispecific antibody, wherein the multispecific antibody comprises a second antibody moiety that specifically binds to a second antigen. In certain embodiments, the second antigen is a tumor associated antigen. In certain embodiments, the tumor associated antigen is selected from the group consisting of Her-2, EGFR, PDL1, c-Met, B Cell Maturation Antigen (BCMA), carbonic anhydrase IX (CA1X), carcinoembryonic antigen (CEA), CDS, CD7, CD10, CD19, CD20, CD22, CD30, CD33, CD34, CD38, CD41, CD44, CD49f, CD56, CD74, CD123, CD133, CD138, CD276 (B7H3), epithelial glycoprotein (EGP2), trophoblast cell-surface antigen 2 (TROP-2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AChR), folate receptor-a, Ganglioside G2 (GD2), Ganglioside G3 (GD3), human telomerase reverse transcriptase (hTERT), kinase insert domain receptor (KDR), Lewis A (CA 1.9.9), Lewis Y (LeY), B7H3, L1 cell adhesion molecule (L1CAM), Mucin 16 (Muc-16), Mucin 1 (Muc-1), NG2D ligands, oncofetal antigen (h5T4), prostate stem cell antigen (PSCA), prostate-specific membrane antigen (PSMA), tumor-associated glycoprotein 72 (TAG-72), Claudin18.2 (CLDN18.2), vascular endothelial growth factor R2 (VEGF-R2), Wilms tumor protein (WT-1), type 1 tyrosine-protein kinase transmembrane receptor (ROR1), PVR, PVRL2, and any combination thereof. In certain embodiments, the second antigen is an immune checkpoint regulator. In certain embodiments, the immune checkpoint regulator is selected from the group consisting of TIGIT, PD1, CTLA4, LAG-3, 2B4, BTLA and any combination thereof. In certain embodiments, the second antigen is an immune costimulatory molecule or a subunit of a T cell receptor/CD3 complex. In certain embodiments, the immune costimulatory molecule is selected from the group consisting of CD28, ICOS, CD27, 4-1BB, OX40 and CD40 and any combination thereof. In certain embodiments, the subunit of the T cell receptor/CD3 complex is selected from the group consisting of CD3γ, CD3δ, CD3ε and any combination thereof.

The present disclosure provides an immunoconjugate comprising any antibody disclosed herein, linked to a therapeutic agent or a label. In certain embodiments, the therapeutic agent is a cytotoxin or a radioactive isotope. In certain embodiments, the label is selected from the group consisting of a radioisotope, a fluorescent dye and an enzyme.

The present disclosure provides a chimeric antigen receptor (CAR) comprising an extracellular antigen-binding domain that comprises an antibody disclosed herein. In certain embodiments, the antibody is a VHH.

The present disclosure provides an immunoresponsive cell comprising a CAR disclosed herein. In certain embodiments, the immunoresponsive cell is selected from the group consisting of a T cell, a Natural Killer (NK) cell, a cytotoxic T lymphocyte (CTL), a regulatory T cell, a Natural Killer T (NKT) cell and a myeloid cell. In certain embodiments, the immunoresponsive cell is a T cell.

The present disclosure provides a pharmaceutical composition comprising a) any antibody disclosed herein, any immunoconjugate disclosed herein, or any immunoresponsive cell disclosed herein, and b) a pharmaceutically acceptable carrier.

The present disclosure further provides a nucleic acid encoding any antibody disclosed herein, a vector comprising any nucleic acid disclosed herein, and a host cell comprising any nucleic acid or vector disclosed herein.

The present disclosure provides a method for preparing an antibody disclosed herein comprising expressing the antibody in a host cell disclosed herein and isolating the antibody from the host cell.

The present disclosure further provides a method of reducing tumor burden in a subject. In certain embodiments, the method comprising administering to the subject an effective amount of an antibody disclosed herein, an immunoconjugate disclosed herein, or a pharmaceutical composition disclosed herein. In certain embodiments, the method reduces the number of tumor cells. In certain embodiments, the method reduces tumor size. In certain embodiments, the method eradicates the tumor in the subject. In certain embodiments, the tumor exhibits high microsatellite instability (MSI). In certain embodiments, the tumor is selected from the group consisting of mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, cholangiocarcinoma, head and neck cancer, blood cancer and a combination thereof.

The present disclosure provides methods of treating and/or preventing cancer, or lengthening survival of a subject having cancer. In certain embodiments, the method comprising administering to the subject an effective amount of an antibody disclosed herein, an immunoconjugate disclosed herein, or a pharmaceutical composition disclosed herein. In certain embodiments, the cancer exhibits high microsatellite instability (MSI). In certain embodiments, the cancer is selected from the group consisting of mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, cholangiocarcinoma, head and neck cancer, blood cancer and a combination thereof.

The present disclosure further provides any antibody and/or pharmaceutical composition disclosed herein for use as a medicament. The present disclosure further provides any antibody and/or pharmaceutical composition disclosed herein for use in treating cancer. In certain embodiments, the cancer exhibits high microsatellite instability (MSI). In certain embodiments, the cancer is selected from the group consisting of mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, cholangiocarcinoma, head and neck cancer, blood cancer and a combination thereof.

The present disclosure provides a kit comprising an antibody disclosed herein, an immunoconjugate disclosed herein, a pharmaceutical composition disclosed herein, a nucleic acid disclosed herein, a vector disclosed herein or an immunoresponsive cell disclosed herein.

In certain embodiments, the kit further comprises a written instruction for treating and/or preventing a neoplasm.

### BRIEF DESCRIPTION OF THE FIGURES

FIGS. 1A and 1B depict schematics of GPC3 molecule and an exemplary anti-GPC3 VHH antibody disclosed herein, respectively. FIG. 1A depicts a schematic of the structure of human GPC3 molecule, which consists of 580 amino acids and two heparan sulfate (HS) side chains attached close to the C-terminal portion. GPC3 can be cleaved by furin enzyme between Arg358 and Cys359, resulting in a 40-kDa N-terminal subunit and a 30-kDa C-terminal subunit linked by a disulfide bond. FIG. 1B depicts schematics of the structure of a llama derived VHH-Fc antibody (left panel) and the structural model of the VHH (right panel).

FIG. 2 depicts the binding ability of the two top VHH-Fc clones to HepG2 hepatoma cell line by flow cytometry. HN3 VHH-Fc was used as a positive control.

FIGS. 3A-3D depict the binding activity of 1B01 VHH-Fc, HN3 VHH-Fc and afycosylated (AF) 1B01 VHH-Fc to human GPC3 (3A), cynomolgus monkey GPC3 (3B), mouse GPC3 (3C) and human GPC3 C-terminal domain (3D) measured by ELISA.

FIGS. 4A and 4B depict whole cell binding of anti-GPC3 antibodies to HepG2 (4A) and Hep3B hepatoma cells (4B) measured by flow cytometry.

FIG. 5 depicts antibody-dependent cell-mediated cytotoxicity (ADCC) activity of the anti-GPC3 antibodies measured by percent cell lysis using human PBMCs as effector cells and HeG2 hepatoma cells as target cell.

FIG. 6 depicts tumor growth curves in a HepG2 hepatoma mouse model under the treatment of anti-GPC3 VHH antibodies or a vehicle control.

### DETAILED DESCRIPTION

The present disclosure provides isolated monoclonal antibodies and antibody derivatives that bind specifically to GPC3 with high affinity, including monospecific anti-GPC3 antibodies and multispecific antibodies that binds to GPC3 and one or more additional target. In certain embodiments, an antibody or antibody derivative disclosed herein comprises a single domain antibody that binds to GPC3. This disclosure further provides methods of making and using antibodies and antibody derivatives disclosed herein and pharmaceutical compositions comprising the same, e.g., for treating diseases and disorders, e.g., cancer. The invention is based, in part, on the discovery of novel single domain antibodies that bind to GPC3, which can target a tumor cell and/or increase an immune response against a tumor cell and thereby provide improved anti-tumor efficacy.

For clarity and not by way of limitation the detailed description of the presently disclosed subject matter is divided into the following subsections:
1. Definitions;
2. Antibodies and antibody derivatives;
3. Methods of use;
4. Pharmaceutical formulations; and
5. Articles of manufacture.

### 1. Definitions

The term “antibody” as referred to herein includes full-length antibodies and any antigen-binding fragment thereof (i.e., antibody fragment). An “antibody” can be a standalone molecule or a portion of an antibody derivative. Exemplary antibody derivatives include, but are not limited to, a multispecific antibody (e.g., a bispecific antibody), an antigen-recognizing receptor (e.g., a chimeric antigen receptor), an antibody conjugate comprising an additional proteinaceous or non-proteinaceous moiety (e.g., an antibody-drug conjugate or a polymer-coated antibody), and other multifuctional molecules comprising an antibody.

A “full-length antibody”, “intact antibody” and “whole antibody” refers to an antibody similar to a native antibody structure or having heavy chains that contain an Fc region as defined herein. In certain embodiments, a full-length antibody comprises two heavy chains and two light chains. In certain embodiments, the variable regions of the light and heavy chains are responsible for antigen binding. The variable regions of a heavy chain and a light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by well-known conventions, e.g., the conventions of Kabat, Chothia, MacCallum, IMGT and AHo as described below. The three CDRs of the heavy or light chains are interposed between flanking stretches known as framework regions (FRs), which are more conserved than the CDRs and form a scaffold to support the hypervariable loops. The constant regions of the heavy and light chains are not involved in antigen binding but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain). In certain embodiments, a full-length antibody is glycosylated. In certain embodiments, a full-length antibody comprises a glycan linked to its Fc region. In certain embodiments, a full-length antibody comprises a branched glycan.

The term “antigen-binding portion”, “antibody fragment” and “antibody portion” of an antibody, as used herein, refers to one or more fragments of an antibody that retain the ability to specifically bind to an antigen. It has been shown that the antigen-binding function of an antibody can be performed by fragments of a full-length antibody. Examples of antibody fragments include but are not limited to Fv, Fab, Fab′, Fab′-SH, F(ab′)2, diabodies, linear antibodies, single-chain antibody molecules (e.g., scFv and scFv-Fc), a single domain antibody, a VHH, a VHH-Fc, a nanobody, a domain antibody, a bivalent domain antibody, or any other fragment or combination thereof of an antibody that binds to an antigen. A “VHH” refers to a single domain antibody isolated from a camelid animal. In certain embodiments, a VHH comprises a variable region of a heavy chain of a camelid heavy chain antibody. In certain embodiments, a VHH has a size of no more than about 25 kDa. In certain embodiments, a VHH has a size of no more than about 20 kDa. In certain embodiments, a VHH has a size of no more than about 15 kDa.

An “antibody that cross-competes for binding” with a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is described in Antibodies, Harlow and Lane (Cold Spring Harbor Press, Cold Spring Harbor, NY).

“Fv” is a minimum antibody fragment which contains a complete antigen-recognition and -binding site. This fragment consists of a dimer of one heavy- and one light-chain variable region in tight, non-covalent association. From the folding of these two domains emanate six hypervariable loops (3 loops in each of the heavy and light chains) that contribute the amino acid residues to antigen binding and confer antigen binding specificity to the antibody. However, even a single variable domain (or half of an Fv comprising only three CDRs specific for an antigen) can recognize and bind to an antigen, although sometimes at a lower affinity than the entire binding site.

“Single-chain Fv”, also abbreviated as “sFv” or “scFv”, are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

An “acceptor human framework” or “human framework” for the purposes herein is a framework comprising the amino acid sequence of a light chain variable domain (VL) framework or a heavy chain variable domain (VH) framework derived from a human immunoglobulin framework or a human consensus framework. An acceptor human framework “derived from” a human immunoglobulin framework or a human consensus framework may comprise the same amino acid sequence thereof, or it may contain amino acid sequence changes. In certain embodiments, the number of amino acid changes are 10 or less, 9 or less, 8 or less, 7 or less, 6 or less, 5 or less, 4 or less, 3 or less, or 2 or less. In certain embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary embodiments for measuring binding affinity are described in the following.

An “affinity matured” antibody refers to an antibody with one or more alterations in one or more CDRs or hypervariable regions (HVRs), compared to a parent antibody which does not possess such alterations, which alterations provide improved affinity of the antibody for antigen.

“GPC3”, “GPC3 protein” or “GPC3 polypeptide” as used herein, refers to any GPC3 polypeptide from any vertebrate source, including mammals such as primates (e.g., humans and cynomolgus monkeys), or any fragment thereof, and may optionally comprise up to one, up to two, up to three, up to four, up to five, up to six, up to seven, up to eight, up to nine or up to ten amino acid substitutions, additions and/or deletions. The term encompasses full-length, unprocessed GPC3 as well as any form of GPC3 that results from processing in the cell. The term also encompasses naturally occurring variants of GPC3, e.g., splice variants or allelic variants. In certain embodiments, a GPC3 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001158089.1, NP_001158090.1, NP_001158091.1, NP_004475.1 or XP_016884902.1 (homology herein may be determined using standard software such as BLAST or FASTA). In certain embodiments, the GPC3 polypeptide comprises or has an amino acid sequence that is the entirety or a consecutive portion of SEQ ID NO: 14.

The term “ECD of GPC3” refers to an extracellular domain of GPC3. In certain embodiments, the ECD is a N-terminal ECD. In certain embodiments, the ECD is a C-terminal ECD. In certain embodiments, the C-terminal ECD of an exemplary GPC3 polypeptide can comprise the amino acid sequence set forth in SEQ ID NO: 15.

The terms “anti-GPC3 antibody” and “an antibody that binds to GPC3” refer to an antibody that is capable of binding to GPC3 with sufficient affinity such that the antibody is useful as a diagnostic and/or therapeutic agent for targeting GPC3. In one embodiment, the extent of binding of an anti-GPC3 antibody to an unrelated, non-GPC3 protein is less than about 10% of the binding of the antibody to GPC3 as measured, e.g., by a BIACORE® surface plasmon resonance assay. In certain embodiments, an antibody that binds to GPC3 has a dissociation constant (KD) of <about 1 μM, <about 100 nM, <about 10 nM, <about 1 nM, <about 0.1 nM, <about 0.01 nM, or <about 0.001 nM (e.g., 10⁻⁸ M or less, e.g., from 10⁻⁸ M to 10⁻¹² M, e.g., from 10⁻⁹ M to 10⁻¹⁰ M). In certain embodiments, an anti-GPC3 antibody binds to an epitope of GPC3 that is conserved among GPC3 from different species. In certain embodiments, an anti-GPC3 antibody binds to an epitope on GPC3 that is in the ECD of the protein. In certain embodiments, an anti-GPC3 antibody binds to an epitope on GPC3 that is in the C-terminal ECD of the protein.

The term “chimeric” antibody refers to an antibody in which a portion of the heavy and/or light chain is derived from a particular source or species, while the remainder of the heavy and/or light chain is derived from a different source or species. In certain embodiments, a chimeric antibody disclosed herein comprises a camelid heavy chain variable region and a human Fc region.

As used herein, the term “CDR” or “complementarity determining region” is intended to mean the non-contiguous antigen combining sites within the variable region of a heavy chain and/or a light chain. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of any one of the definitions to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR definitions

| | Kabat¹ | Chothia² | MacCallum³ | IMGT⁴ | AHO⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra.
²Residue numbering follows the nomenclature of Chothia et al., supra.
³Residue numbering follows the nomenclature of MacCallum et al., supra.
⁴Residue numbering follows the nomenclature of Lefranc et al., supra.
⁵Residue numbering follows the nomenclature of Honegger and Plückthun, supra.

The expression “variable-domain residue-numbering as in Kabat” or “amino-acid-position numbering as in Kabat”, and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or CDR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

In certain embodiments, the amino acid residues which encompass the CDRs of a single domain antibody (e.g., a single domain anti-GPC3 antibody disclosed herein) is defined according to the IMGT nomenclature in Lefranc et al., supra. In certain embodiments, the amino acid residues which encompass the CDRs of a full-length antibody is defined according to the Kabat nomenclature in Kabat et al., supra. In certain embodiments, the numbering of the residues in an immunoglobulin heavy chain, e.g., in an Fc region, is that of the EU index as in Kabat et al., supra. The “EU index as in Kabat” refers to the residue numbering of the human IgG1 EU antibody.

“Framework” or “FR” refers to residues are those variable-domain residues other than the CDR residues as herein defined.

A “humanized” antibody refers to a chimeric antibody comprising amino acid residues from non-human CDRs/HVRs and amino acid residues from human FRs. In certain embodiments, a humanized antibody will comprise substantially all of at least one, and typically two, variable domains, in which all or substantially all of the HVRs/CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the C_{L} domain of the light chain.

The “light chains” of antibodies (e.g., immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” domain) comprises the residues between a CH2 domain and the C-terminal of an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibitory receptor FcγRIIB contains an immunoreceptor tyrosine-based inhibition motif (ITIM) in its cytoplasmic domain. (See M. Daeron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “epitope” as used herein refers to the specific group of atoms or amino acids on an antigen to which an antibody or antibody derivative binds. Two antibodies or antigen-binding moieties may bind the same epitope within an antigen if they exhibit competitive binding for the antigen.

As use herein, the terms “specifically binds”, “specifically recognizing”, and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, greater avidity, greater readiness, and/or greater duration than its bindings to other targets. In some embodiments, the extent of binding of an antibody to an unrelated target is less than about 10% of the binding of the antibody to the target as measured, e.g., by a radioimmunoassay (MA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, MA-, ECL-, IRMA-, EIA-, BIACORE™-tests and peptide scans.

An “isolated” antibody (or construct) is one that has been identified, separated and/or recovered from a component of its production environment (e.g., natural or recombinant). In certain embodiments, the isolated polypeptide is free or substantially free from association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. In certain embodiments, the isolated nucleic acid is free or substantially free from association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid, which cell includes the primary subject cell and its progeny.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell and may contain mutations. Mutant progeny that have the same function or biological activity as screened or selected for in the originally transformed cell are included herein.

The terms “subject”, “individual”, and “patient” are used interchangeably herein to refer to a mammal, including, but not limited to, human, bovine, horse, feline, canine, rodent, or primate. In some embodiments, the subject is a human.

An “effective amount” of an agent refers to an amount effective, at dosages and for periods of time necessary, to achieve the desired therapeutic or prophylactic result. The specific dose may vary depending on one or more of the particular agent chosen, the dosing regimen to be followed, whether it is administered in combination with other compounds, timing of administration, the tissue to be imaged, and the physical delivery system in which it is carried.

A “therapeutically effective amount” of a substance/molecule of the application, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment. “Treatment” does not necessarily mean that the condition being treated will be cured.

It is understood that embodiments of the application described herein include “consisting” and/or “consisting essentially of” embodiments.

As used herein, the term “about” or “approximately” means within an acceptable error range for the particular value as determined by one of ordinary skill in the art, which will depend in part on how the value is measured or determined, i.e., the limitations of the measurement system. In certain embodiments, “about” can mean within 3 or more than 3 standard deviations, per the practice in the art. In certain embodiments, “about” can mean a range of up to 20%, e.g., up to 10%, up to 5%, or up to 1% of a given value. In certain embodiments, particularly with respect to biological systems or processes, the term can mean within an order of magnitude, e.g., within 5-fold or within 2-fold, of a value.

As used herein, the term “modulate” means positively or negatively alter. Exemplary modulations include a about 1%, about 2%, about 5%, about 10%, about 25%, about 50%, about 75%, or about 100% change.

As used herein, the term “increase” means alter positively by at least about 5%. An alteration may be by about 5%, about 10%, about 25%, about 30%, about 50%, about 75%, about 100% or more.

As used herein, the term “reduce” means alter negatively by at least about 5%. An alteration may be by about 5%, about 10%, about 25%, about 30%, about 50%, about 75%, or even by about 100%.

The term “about X-Y” used herein has the same meaning as “about X to about Y.”

As used herein and in the appended claims, the singular forms “a”, “or”, and “the” include plural referents unless the context clearly dictates otherwise.

“Effector functions” refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), phagocytosis, down regulation of cell surface receptors (e.g., B cell receptor), and B cell activation.

An “immunoconjugate” refers to an antibody conjugated to one or more heterologous molecule(s), including but not limited to a cytotoxic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier”, as used herein, refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “variable region” or “variable domain” refers to the domain of an antibody heavy or light chain that is involved in binding the antibody to antigen. In certain embodiments, the variable domains of the heavy chain and light chain (VH and VL, respectively) of a native antibody generally have similar structures, with each domain comprising four conserved framework regions (FRs) and three CDRs. (See, e.g., Kindt et al. Kuby Immunology, 61ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “antigen-recognizing receptor” as used herein refers to a receptor that is capable of activating an immunoresponsive cell (e.g., a T-cell) in response to its binding to an antigen. Non-limiting examples of antigen-recognizing receptors include native and modified T cell receptors (“TCRs”) and chimeric antigen receptors (“CARs”).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises an antibody or an antibody fragment, e.g., a VHH or a scFv. In certain embodiments, the antibody (e.g., VHH or scFv) is fused to the transmembrane domain, which is fused to the intracellular signaling domain. In certain embodiments, the CAR is selected to have high binding affinity or avidity for the antigen.

By “immunoresponsive cell” is meant a cell that functions in an immune response or a progenitor or progeny thereof.

### 2. Antibodies and Antibody Derivatives

The present disclosure provides isolated monoclonal antibodies and antibody derivatives, including monospecific anti-GPC3 antibodies and multispecific antibodies that binds to GPC3 and one or more additional target. In certain embodiments, an antibody or antibody derivative disclosed herein comprises a single domain antibody that binds to GPC3. In certain embodiments, the disclosure is based, in part, on the discovery of single domain antibodies that bind to GPC3, which can be used in antitumor therapeutics where the antibodies selectively target a tumor cell and/or inhibit a signal pathway mediated by GPC3 and thereby induce beneficial anti-tumor effects against a tumor cell. In certain embodiments, the single domain antibody disclosed herein is an antagonist antibody, which inhibits GPC3 functions. In certain embodiments, the single domain antibody can enhance an antitumor immune response against a tumor cell that expresses a GPC3 protein. In certain embodiments, the single domain antibody comprises a camelid antibody or a VHH antibody. In certain embodiments, the single domain antibody has an improved capability of tissue infiltration due to its smaller size compared to traditional antibodies in the forms of IgG, Fab and/or scFv.

In certain embodiments, an antibody of the present disclosure can be or comprise a monoclonal antibody, including a chimeric, humanized or human antibody. In certain embodiments, the antibody disclosed herein comprises a humanized antibody. In certain embodiments, the antibody comprises an acceptor human framework, e.g., a human immunoglobulin framework or a human consensus framework.

In certain embodiments, an antibody of the present disclosure can be an antibody fragment, e.g., a Fv, Fab, Fab′, scFv, diabody, or F(ab′)2 fragment. In certain embodiments, the antibody is a full-length antibody, e.g., an intact IgG1 antibody, or other antibody class or isotype as defined herein. In certain embodiments, an antibody or antibody derivative of the present disclosure can incorporate any of the features, singly or in combination, as described in this application, e.g., Sections 2.1-2.12 detailed herein.

Antibodies and antibody derivatives of the present disclosure are useful, e.g., for the diagnosis or treatment of a neoplasm or a cancer. In certain embodiments, the neoplasia and cancers whose growth may be inhibited using the antibodies of this disclosure include neoplasia and cancers typically responsive to immunotherapy. In certain embodiments, the neoplasia and cancers include breast cancer (e.g., breast cell carcinoma), ovarian cancer (e.g., ovarian cell carcinoma) and renal cell carcinoma (RCC). Examples of other cancers that may be treated using the methods of this disclosure include melanoma (e.g., metastatic malignant melanoma), prostate cancer, colon cancer, lung cancer, bone cancer, pancreatic cancer, skin cancer, brain tumors, chronic or acute leukemias including acute myeloid leukemia, chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma) nasopharangeal carcinomas, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the breast gland, sarcoma of soft tissue, cancer of the urethra, cancer of the penis, solid tumors of childhood, cancer of the bladder, cancer of the kidney or ureter, carcinoma of the breast pelvis, neoplasm of the central nervous system (CNS), tumor angiogenesis, spinal axis tumor, brain stem glioma, pituitary adenoma, Kaposi's sarcoma, epidermoid cancer, squamous cell cancer, environmentally induced cancers including those induced by asbestos, e.g., mesothelioma and combinations of said cancers.

### 2.1 Exemplary Anti-GPC3 Antibodies

The present disclosure provides isolated antibodies that bind to a GPC3 protein. In certain embodiments, an anti-GPC3 antibody of the present disclosure binds to the ECD of GPC3. In certain embodiments, the anti-GPC3 antibody binds to the C-terminal ECD of GPC3. In certain embodiments, the C-terminal ECD comprises the amino acid sequence set forth in SEQ ID NO: 15. In certain embodiments, the anti-GPC3 antibody binds to the same epitope with an anti-GPC3 antibody described herein, e.g., 1B01.

In certain embodiments, the anti-GPC3 antibody disclosed herein can function as an antagonist of a GPC3-based signal pathway. In certain embodiments, the anti-GPC3 antibody can block or reduce a signal pathway that depends on a GPC3 protein. In certain embodiments, the anti-GPC3 antibody can reduce the activity of the signal pathway by at least about 10%, about 20%, about 30%, about 40%, about 50%, about 60%, about 70%, about 80%, about 90%, about 99% or about 99.9%. In certain embodiments, treatment using the anti-GPC3 antibody exhibits antitumor efficacy in a subject, whereby reduces tumor growth and/or lengthen the survival of a subject. In certain embodiments, the anti-GPC3 antibody increases an immune response and/or an antitumor effect of an immune cell, e.g., a T cell and/or a NK cell against a tumor cell that expresses GPC3. In certain embodiments, the anti-GPC3 antibody comprising a single domain antibody (e.g., a VHH) has a smaller molecule size compared to a full-length antibody due to the smaller size of a single domain antibody compared to a Fab domain of a full-length antibody, which can result in superior tissue infiltration, e.g., at a tumor site, compared to a full-length antibody. In certain embodiments, treatment using the anti-GPC3 antibody exhibits superior antitumor efficacy compared to treatment using a full-length anti-GPC3 antibody.

In certain embodiments, the anti-GPC3 antibody comprises a single domain antibody that binds to GPC3. In certain embodiments, the single domain antibody comprises a VHH. In certain embodiments, the single domain antibody comprises a heavy chain variable region (VH). In certain embodiments, the single domain antibody is linked to a Fc region. In certain embodiments, the single domain antibody is not linked to a Fc region.

In certain embodiments, the single domain antibody binds to GPC3 with a KD of about 1×10⁻⁷ M or less. In certain embodiments, the single domain antibody binds to GPC3 with a KD of about 1×10⁻⁸ M or less. In certain embodiments, the single domain antibody binds to GPC3 with a KD of about 5×10⁻⁹ M or less. In certain embodiments, the single domain antibody binds to GPC3 with a KD of about 1×10⁻⁹ M or less. In certain embodiments, the single domain antibody binds to GPC3 with a KD of about 1×10⁻¹⁰ M or less. In certain embodiments, the single domain antibody binds to GPC3 with a KD of between about 1×10⁻¹¹ M and about 1×10⁻⁷ M. In certain embodiments, the single domain antibody binds to GPC3 with a KD of between about 1×10⁻¹⁰ M and about 1×10⁻⁷ M. In certain embodiments, the single domain antibody binds to GPC3 with a KD of between about 1×10⁻¹⁰ M and about 1×10⁻⁸ M. In certain embodiments, the single domain antibody binds to GPC3 with a KD of between about 1×10⁻¹¹ M and about 1×10⁻⁹ M. In certain embodiments, the single domain antibody binds to GPC3 with a KD of between about 2×10⁻¹⁰ M and about 5×10⁻⁹ M. In certain embodiments, the single domain antibody binds to GPC3 with a KD of between about 1×10⁻⁹ M and about 5×10⁻⁸ M. In certain embodiments, the single domain antibody binds to GPC3 with a KD of between about 1×10⁻¹⁰ M and about 1×10⁻⁹ M.

In certain embodiments, the single domain antibody cross-competes for binding to GPC3 with a reference anti-GPC3 single domain antibody comprising a heavy chain variable region CDR1 comprising the amino acid sequence set forth in SEQ ID NO: 1, a heavy chain variable region CDR2 comprising the amino acid sequence set forth in SEQ ID NO: 2, and a heavy chain variable region CDR3 comprising the amino acid sequence set forth in SEQ ID NO: 3. In certain embodiments, the single domain antibody cross-competes for binding to GPC3 with a reference anti-GPC3 single domain antibody comprising a heavy chain variable region CDR1 comprising the amino acid sequence set forth in SEQ ID NO: 5, a heavy chain variable region CDR2 comprising the amino acid sequence set forth in SEQ ID NO: 6, and a heavy chain variable region CDR3 comprising the amino acid sequence set forth in SEQ ID NO: 7.

In certain embodiments, the single domain antibody comprises a heavy chain variable region comprising: a) a heavy chain variable region CDR1 comprises an amino acid sequence of any one of SEQ ID NOs: 1 and 5, or a variant thereof comprising up to about 3 amino acid substitutions; b) a heavy chain variable region CDR2 comprises an amino acid sequence of any one of SEQ ID NOs: 2 and 6, or a variant thereof comprising up to about 3 amino acid substitutions; and c) a heavy chain variable region CDR3 comprises an amino acid sequence of any one of SEQ ID NOs: 3 and 7, or a variant thereof comprising up to about 3 amino acid substitutions.

In certain embodiments, the single domain antibody comprises a heavy chain variable region that comprises a CDR1 domain, a CDR2 domain and a CDR3 domain, wherein the CDR1 domain, the CDR2 domain and the CDR3 domain respectively comprise a CDR1 domain, a CDR2 domain and a CDR3 domain comprised in a reference heavy chain variable region comprising the amino acid sequence selected from the group consisting of SEQ ID NOs: 4, 8 and 12.

In certain embodiments, the single domain antibody comprises a heavy chain variable region CDR1 comprising the amino acid sequence set forth in SEQ ID NO: 1, a heavy chain variable region CDR2 comprising the amino acid sequence set forth in SEQ ID NO: 2, and a heavy chain variable region CDR3 comprising the amino acid sequence set forth in SEQ ID NO: 3. In certain embodiments, the single domain antibody comprises a heavy chain variable region CDR1 comprising the amino acid sequence set forth in SEQ ID NO: 5, a heavy chain variable region CDR2 comprising the amino acid sequence set forth in SEQ ID NO: 6, and a heavy chain variable region CDR3 comprising the amino acid sequence set forth in SEQ ID NO: 7.

In certain embodiments, the single domain antibody comprises a heavy chain variable region comprising an amino acid sequence having at least about 80%, 85%, 90%, 91%, 92%, 93%, 94%, 95%, 96%, 97%, 98%, 99%, or 100% sequence identity to the amino acid sequence selected from the group consisting of SEQ ID NOs: 4, 8 and 12. In certain embodiments, the single domain antibody comprises a heavy chain variable region comprising an amino acid sequence selected from the group consisting of SEQ ID NOs: 4, 8 and 12.

In certain embodiments, the single domain antibody comprises a heavy chain variable region comprising the amino acid sequence set forth in SEQ ID NO: 4. In certain embodiments, the single domain antibody comprises a heavy chain variable region comprising the amino acid sequence set forth in SEQ ID NO: 8. In certain embodiments, the single domain antibody comprises a heavy chain variable region comprising the amino acid sequence set forth in SEQ ID NO: 12.

In certain embodiments, any one of the amino acid sequences comprised in the heavy chain variable region can comprise up to about 1, about 2, about 3, about 4, about 5, about 6, about 7, about 8, about 9 or about 10 amino acid substitutions, deletions and/or additions. In certain embodiments, the amino acid substitution is a conservative substitution.

In certain embodiments, the single domain antibody comprises a humanized framework. In certain embodiments, the humanized framework comprises a framework sequence of the heavy chain variable region sequence set forth in SEQ ID NO: 12.

In certain embodiments, the anti-GPC3 antibody does not comprise a Fc region. In certain embodiments, the anti-GPC3 antibody further comprises a Fc region. In certain embodiments, the Fc region comprises a human Fc region. In certain embodiments, the Fc region comprises a Fc region selected from the group consisting of the Fc regions of IgG, IgA, IgD, IgE and IgM. In certain embodiments, the Fc region comprises a Fc region selected from the group consisting of the Fc regions of IgG1, IgG2, IgG3 and IgG4. In certain embodiments, the Fc region comprises an IgG1 Fc region. In certain embodiments, the IgG1 Fc region comprising one or more mutation that modifies an antibody-dependent cell-mediated cytotoxicity (ADCC). In certain embodiments, the IgG1 Fc region comprising one or more mutation that reduces an antibody-dependent cell-mediated cytotoxicity (ADCC). In certain embodiments, the IgG1 Fc region comprising one or more mutation that enhances an antibody-dependent cell-mediated cytotoxicity (ADCC). In certain embodiments, the IgG1 Fc region comprises the mutations of L235V, F243L, R292P, Y300L and P396L. In certain embodiments, the IgG1 Fc region comprises the mutations of S239D, A330L and I332E. In certain embodiments, the anti-GPC3 antibody comprises the amino acid sequence set forth in SEQ ID NO: 13.

In certain embodiments, the heavy chain variable region is linked to a Fc region via a linker. In certain embodiments, the linker is a peptide linker. In certain embodiments, the peptide linker comprises about four to about thirty amino acids. In certain embodiments, the peptide linker comprises about four to about fifteen amino acids. In certain embodiments, the peptide linker comprise an amino acid sequence selected from the group consisting of SEQ ID NOs: 16-50.

In certain embodiments, the anti-GPC3 antibody comprises a full-length immunoglobulin, a single-chain Fv (scFv) fragment, a Fab fragment, a Fab′ fragment, a F(ab′)2, an Fv fragment, a disulfide stabilized Fv fragment (dsFv), a (dsFv)2, a VHH, a Fv-Fc fusion, a scFv-Fc fusion, a VHH-Fv fusion, a diabody, a tribody, a tetrabody or any combination thereof.

In certain embodiments, the antibody is comprised in a larger molecule that is an antibody derivative. In certain embodiments, the antibody derivative is a multispecific antibody, e.g., a bispecific antibody, wherein the multispecific antibody comprises a second antibody moiety that specifically binds to a second antigen. In certain embodiments, the second antigen is a tumor associated antigen. In certain embodiments, the tumor associated antigen is selected from the group consisting of Her-2, EGFR, PD-L1, MSLN, c-Met, B Cell Maturation Antigen (BCMA), carbonic anhydrase IX (CA1X), carcinoembryonic antigen (CEA), CDS, CD7, CD10, CD19, CD20, CD22, CD30, CD33, CD34, CD38, CD41, CD44, CD47, CD49f, CD56, CD74, CD123, CD133, CD138, CD276 (B7H3), epithelial glycoprotein (EGP2), trophoblast cell-surface antigen 2 (TROP-2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AChR), folate receptor-a, Ganglioside G2 (GD2), Ganglioside G3 (GD3), human telomerase reverse transcriptase (hTERT), kinase insert domain receptor (KDR), Lewis A (CA 1.9.9), Lewis Y (LeY), L1 cell adhesion molecule (L1CAM), Mucin 16 (Muc-16), Mucin 1 (Muc-1), NG2D ligands, oncofetal antigen (h5T4), prostate stem cell antigen (PSCA), prostate-specific membrane antigen (PSMA), tumor-associated glycoprotein 72 (TAG-72), Claudin18.2 (CLDN18.2), vascular endothelial growth factor R2 (VEGF-R2), Wilms tumor protein (WT-1), type 1 tyrosine-protein kinase transmembrane receptor (ROR1), PVR, PVRL2 and any combination thereof. In certain embodiments, the second antigen is an immune checkpoint regulator. In certain embodiments, the immune checkpoint regulator is selected from the group consisting of TIGIT, PD1, CTLA4, LAG-3, 2B4, BTLA and any combination thereof. In certain embodiments, binding of the antibody derivative or multispecific antibody to the second antigen inhibits the immune checkpoint regulator. In certain embodiments, the second antigen is an immune costimulatory molecule or a subunit of a T cell receptor/CD3 complex. In certain embodiments, the immune costimulatory molecule is selected from the group consisting of CD28, ICOS, CD27, 4-1BB, OX40, CD40 and any combination thereof. In certain embodiments, binding of the antibody derivative or multispecific antibody to the second antigen activates the immune costimulatory molecule. In certain embodiments, the subunit of the T cell receptor/CD3 complex is selected from the group consisting of CD3γ, CD3δ, CD3ε and any combination thereof. In certain embodiments, binding of the antibody derivative or multispecific antibody to the second antigen activates the T cell receptor/CD3 complex.

In certain embodiments, the anti-GPC3 antibody is linked to the second antigen binding moiety via a linker. In certain embodiments, the linker is a peptide linker. In certain embodiments, the peptide linker comprises about four to about thirty amino acids. In certain embodiments, the peptide linker comprises about four to about fifteen amino acids. In certain embodiments, the peptide linker comprises an amino acid sequence selected from the group consisting of SEQ ID NOs: 16-50.

In certain embodiments, the anti-GPC3 antibody is conjugated to a therapeutic agent or a label. In certain embodiments, the label is selected from the group consisting of a radioisotope, a fluorescent dye and an enzyme.

### 2.2 Antibody Affinity

In certain embodiments, an antibody or antibody derivative disclosed herein has a high binding affinity to its target antigen. In certain embodiments, the antibody or antibody derivative binds to the target with a KD of about 1×10⁻⁷ M or less. In certain embodiments, the antibody or antibody derivative binds to the target with a KD of about 1×10⁻⁸ M or less. In certain embodiments, the antibody or antibody derivative binds to the target with a KD of about 5×10⁻⁹ M or less. In certain embodiments, the antibody or antibody derivative binds to the target with a KD of about 1×10⁻⁹ M or less. In certain embodiments, the antibody or antibody derivative binds to the target with a KD of about 1×10⁻¹⁰ M or less.

In certain embodiments, the antibody or antibody derivative binds to the target with a KD of between about 1×10⁻¹¹ M and about 1×10⁻⁷ M. In certain embodiments, the antibody or antibody derivative binds to the target with a KD of between about 1×10⁻¹⁰ M and about 1×10⁻⁷ M. In certain embodiments, the antibody or antibody derivative binds to the target with a KD of between about 1×10⁻¹⁰ M and about 1×10⁻⁸ M. In certain embodiments, the antibody or antibody derivative binds to the target with a KD of between about 1×10⁻¹¹ M and about 1×10⁻⁹ M. In certain embodiments, the antibody or antibody derivative binds to the target with a KD of between about 2×10⁻¹⁰ M and about 5×10⁻⁹ M. In certain embodiments, the antibody or antibody derivative binds to the target with a KD of between about 1×10⁻⁹ M and about 5×10⁻⁸ M. In certain embodiments, the antibody or antibody derivative binds to the target with a KD of between about 1×10⁻¹⁰ M and about 1×10⁻⁹ M.

The KD of the antibody or antibody derivative can be determined by methods known in the art. Such methods comprise, but are not limited to Western blots, ELISA-, RIA-, ECL-, IRMA-, EIA-, Octet-BIACORE®-tests and peptide scans.

In certain embodiments, KD can be measured using a BIACORE® surface plasmon resonance assay. For example, and not by way of limitation, an assay using a BIACORE®-2000 or a BIACORE® 3000 (Biacore, Inc., Piscataway, NJ) is performed at 25° C. with immobilized antigen CMS chips at about 10 response units (RU). In certain embodiments, carboxymethylated dextran biosensor chips (CMS, Biacore, Inc.) are activated with N-ethyl-N′-(3- dimethylaminopropyl)-carbodiimide hydrochloride (EDC) and N-hydroxysuccinimide (NETS) according to the supplier's instructions. Antigen is diluted with 10 mM sodium acetate, pH 4.8, to 5 μg/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (KD) can be calculated as the ratio koff/kon. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2.3 Antibody Fragments

In certain embodiments, an antibody of the present disclosure comprises an antigen-binding fragment or antibody fragment. Antibody fragments include, but are not limited to, Fab, Fab′, Fab′-SH, F(ab′)2, VHH, Fv, and scFv fragments, and other fragments described herein. For a review of certain antibody fragments, see Hudson et al. Nat. Med. 9: 129-134 (2003). For a review of scFv fragments, see e.g., Pluckthtin, in The Pharmacology of Monoclonal Antibodies, vol. 113, Rosenburg and Moore eds., (Springer- Verlag, New York), pp. 269-31 5 (1994); see also WO 93/16185; and U.S. Pat. Nos. 5,571,894 and 5,587,458. For discussion of Fab and F(ab)₂ fragments comprising salvage receptor binding epitope residues and having increased in vivo half-life, see U.S. Pat. No. 5,869,046.

In certain embodiments, an antibody of the present disclosure can be a diabody. Diabodies are antibody fragments with two antigen-binding sites that may be bivalent or bispecific. See, for example, EP 404,097; WO 1993/01 161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9: 129-134 (2003).

In certain embodiments, an antibody of the present disclosure can comprise a single domain antibody. Single domain antibodies are antibody fragments that comprise all or a portion of the heavy chain variable domain or all or a portion of the light chain variable domain of an antibody. In certain embodiments, the single domain antibody is a human single-domain antibody (Domantis, Inc., Waltham, MA; see, e.g., U.S. Pat. No. 6,248,516 B1). In certain embodiments, the single domain antibody is camelid single-domain antibody. In certain embodiments, the single domain antibody is a VHH. In certain embodiments, the single domain antibody is humanized.

Antibody fragments can be made by various techniques including, but not limited to, proteolytic digestion of an intact antibody as well as production by recombinant host cells (e.g., E. coli or phage), as described herein.

### 2.4 Chimeric and Humanized Antibodies

In certain embodiments, an antibody of the present disclosure is a chimeric antibody. Certain chimeric antibodies are described, e.g., in U.S. Pat. No. 4,816,567; and Morrison et al., Proc. Natl. Acad. Sci. USA, 81:6851-6855 (1984)). In certain embodiments, a chimeric antibody comprises a non-human variable region (e.g., a variable region derived from mouse) and a human constant region. In certain embodiments, a chimeric antibody is a “class switched” antibody in which the class or subclass has been changed from that of the parent antibody. Chimeric antibodies include antigen-binding fragments thereof.

In certain embodiments, an antibody of the present disclosure can be a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and one or more framework (FR) (or any portion thereof) are derived from human antibody sequences. A humanized antibody optionally can also comprise at least a portion of a human constant region. In certain embodiments, certain FR residues in a humanized antibody are substituted with corresponding residues from a non-human antibody (e.g., the antibody from which the HVR residues are derived), e.g., to restore or improve antibody specificity or affinity.

Humanized antibodies and methods of making them are described, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 2.5 Human Antibodies

In certain embodiments, an antibody of the present disclosure can be a human antibody (e.g., human domain antibody, or human DAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

### 2.6 Library-Derived Antibodies

An antibody of the present disclosure may be isolated by screening combinatorial libraries for antibodies with the desired activity or activities. For example, a variety of methods are known in the art for generating phage display libraries and screening such libraries for antibodies possessing the desired binding characteristics. Such methods are described, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). Methods for constructing single-domain antibody libraries have been described, for example, see U.S. Pat. No. 7,371,849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 2.7 Antibody Variants

The presently disclosure further provides amino acid sequence variants of the disclosed antibodies. For example, it may be desirable to improve the binding affinity and/or other biological properties of the antibody. Amino acid sequence variants of an antibody can be prepared by introducing appropriate modifications into the nucleotide sequence encoding the antibody, or by peptide synthesis. Such modifications include, but are not limited to, deletions from, and/or insertions into and/or substitutions of residues within the amino acid sequences of the antibody. Any combination of deletion, insertion, and substitution can be made to arrive at the final construct, provided that the final antibody, i.e., modified, possesses the desired characteristics, e.g., antigen-binding.

### 2.7.1 Substitution, Insertion, and Deletion Variants

In certain embodiments, antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| Original | Exemplary Substitutions | Preferred |
|---|---|---|
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| Leu (L) | Norleucine; Ile; Val; Met; | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. In certain embodiments, non-conservative substitutions will entail exchanging a member of one of these classes for another class.

In certain embodiments, a type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR (or CDR) residues are mutated and the variant antibodies displayed on phage and screened for a particular biological activity (e.g. binding affinity).

Alterations (e.g., substitutions) may be made in HVRs (or CDRs), e.g., to improve antibody affinity. Such alterations may be made in HVR (or CDRs) “hotspots”, i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In certain embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR (or CDRs)-directed approaches, in which several HVR (or CDRs) residues (e.g., 4-6 residues at a time) are randomized. HVR (or CDRs) residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs (or CDRs) so long as such alterations do not substantially reduce the ability of the antibody to bind antigen. For example, conservative alterations (e.g., conservative substitutions as provided herein) that do not substantially reduce binding affinity may be made in HVRs (or CDRs). Such alterations may be outside of HVR (or CDR) “hotspots” or CDRs. In certain embodiments of the variant VHH sequences provided above, each HVR (or CDR) either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### 2.7.2 Glycosylation Variants

In certain embodiments, an antibody is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody comprises an Fc region (e.g., scFv-Fc), the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In certain embodiments, modifications of the oligosaccharide in the antibody may be made in order to create antibody variants with certain improved properties.

In certain embodiments, the antibody has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al.), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In certain embodiments, the antibody has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### 2.7.3 Fc Region Variants

In certain embodiments, the Fc region of a presently disclosed antibody or antibody derivative may comprise a human Fc region sequence (e.g., a human IgG1, IgG2, IgG3 or IgG4 Fc region) comprising an amino acid modification (e.g. a substitution) at one or more amino acid positions. In certain embodiments, one or more amino acid modifications may be introduced into the Fc region of the antibody moiety (e.g., scFv-Fc or VHH-Fc), thereby generating an Fc region variant.

In certain embodiments, the Fc region possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (Cell Technology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, the Fc region comprises one or more mutation according to EU numbering of residues. In certain embodiments, the Fc region is an IgG1 Fc region. In certain embodiments, the IgG1 Fc region comprises a L234A mutation and/or a L235A mutation. In certain embodiments, the Fc region is an IgG2 or IgG4 Fc region. In certain embodiments, the Fc region is an IgG4 Fc region comprising a F234A, and/or a L235A mutation.

In certain embodiments, the Fc region is an IgG1 Fc region. In certain embodiments, the IgG1 Fc region comprising one or more mutation that modifies an antibody-dependent cell-mediated cytotoxicity (ADCC). In certain embodiments, the IgG1 Fc region comprising one or more mutation that reduces an antibody-dependent cell-mediated cytotoxicity (ADCC). In certain embodiments, the IgG1 Fc region comprising one or more mutation that enhances an antibody-dependent cell-mediated cytotoxicity (ADCC). In certain embodiments, the IgG1 Fc region comprises the mutations of L235V, F243L, R292P, Y300L and P396L. In certain embodiments, the IgG1 Fc region comprises the mutations of S239D, A330L and I332E. In certain embodiments, the IgG1 Fc region comprises the mutations of L235V, F243L, R292P and Y300L. In certain embodiments, the IgG1 Fc region comprises substitutions at positions 298, 333, and/or 334 of the Fc region. In certain embodiments, the IgG1 Fc region comprises the mutations of S267E and L328F.

In certain embodiments, the Fc region comprises an IgG4 Fc region. In certain embodiments, the IgG4 Fc region comprises an S228P mutation.

In certain embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In certain embodiments, the antibody (e.g., scFv-Fc or VHH-Fc) variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### 2.7.4 Cysteine Engineered Antibody Variants

In certain embodiments, it may be desirable to create cysteine engineered antibody moieties, e.g., “thioMAbs”, in which one or more residues of an antibody are substituted with cysteine residues. In certain embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

### 2.8 Antibody Derivatives

In certain embodiments, an antibody described herein may be further modified to be an antibody derivative comprising additional proteinaceous or nonproteinaceous moieties that are known in the art and readily available. Nonproteinaceous moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In certain embodiments, an antibody may be further modified to be an antibody derivative comprising one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In certain embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### 2.9 Methods of Production

The antibodies and antibody derivatives disclosed herein can be produced using any available or known technique in the art. For example, but not by way of limitation, antibodies and antibody derivatives can be produced using recombinant methods and compositions, e.g., as described in U.S. Pat. No. 4,816,567. Detailed procedures to generate antibodies and antibody derivatives are described in the Examples below.

The presently disclosed subject matter further provides isolated nucleic acids encoding an antibody or antibody derivative disclosed herein. For example, the isolated nucleic acid can encode an amino acid sequence comprising the VL and/or an amino acid sequence comprising the VH of the antibody, e.g., the light and/or heavy chains of the antibody.

In certain embodiments, the nucleic acid can be present in one or more vectors, e.g., expression vectors. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the disclosed subject matter is intended to include such other forms of expression vectors, such as viral vectors (e.g., replication defective retroviruses, adenoviruses and adeno- associated viruses) that serve equivalent functions.

Different parts of an antibody or antibody derivative disclosed herein can be constructed in a single, multicistronic expression cassette, in multiple expression cassettes of a single vector, or in multiple vectors. Examples of elements that create polycistronic expression cassette include, but are not limited to, various viral and non-viral Internal Ribosome Entry Sites (IRES, e.g., FGF-1 IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

In certain embodiments, the nucleic acid encoding an antibody or antibody derivative of the present disclosure and/or the one or more vectors including the nucleic acid can be introduced into a host cell. In certain embodiments, the introduction of a nucleic acid into a cell can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. In certain embodiments, a host cell can include, e.g., has been transformed with: a vector comprising a nucleic acid that encodes an amino acid sequence comprising a single domain antibody and/or the VH of a single domain antibody. In certain embodiments, a host cell can include, e.g., has been transformed with: (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In certain embodiments, the host cell is eukaryotic, e.g., a Chinese Hamster Ovary (CHO) cell or lymphoid cell (e.g., YO, NSO, Sp20 cell).

In certain embodiments, the methods of making an antibody or antibody derivative disclosed herein can include culturing a host cell, in which a nucleic acid encoding the antibody or antibody derivative has been introduced, under conditions suitable for expression of the antibody or antibody derivative, and optionally recovering the antibody or antibody derivative from the host cell and/or host cell culture medium. In certain embodiments, the antibody or antibody derivative is recovered from the host cell through chromatography techniques.

For recombinant production of an antibody or antibody derivative of the present disclosure, a nucleic acid encoding an antibody or antibody derivative, e.g., as described above, can be isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody or antibody derivative). Suitable host cells for cloning or expression of antibody-encoding vectors include prokaryotic or eukaryotic cells described herein. For example, an antibody or antibody derivative can be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody or antibody derivative may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody or antibody derivative with a partially or fully human glycosylation pattern. See Gemgross, Nat. Biotech. 22:1409-1414 (2004), and Li et al., Nat. Biotech. 24:21 0-215 (2006). Suitable host cells for the expression of glycosylated antibody can also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. In certain embodiments, plant cell cultures can be utilized as host cells. See, e.g., U.S. Pat. Nos. 5,959,177, 6,040,498, 6,420,548, 7,125,978, and 6,417,429 (describing PLANTIBODIES™ technology for producing antibodies in transgenic plants).

In certain embodiments, vertebrate cells can also be used as hosts. For example, and not by way of limitation, mammalian cell lines that are adapted to grow in suspension can be useful. Non-limiting examples of useful mammalian host cell lines are monkey kidney CV1 line transformed by SY40 (COS-7); human embryonic kidney line (293 or 293 cells as described, e.g., in Graham et al., J Gen Viral. 36:59 (1977)); baby hamster kidney cells (BHK); mouse sertoli cells (TM4 cells as described, e.g., in Mather, Biol. Reprod. 23:243-251 (1980)); monkey kidney cells (CV 1); African green monkey kidney cells (VERO-76); human cervical carcinoma cells (HELA); canine kidney cells (MDCK; buffalo rat liver cells (BRL 3A); human lung cells (W138); human liver cells (Hep 02); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFK CHO cells (Urlaub et al., Proc. Natl. Acad. Sci. USA 77:42 I6 (1980)); and myeloma cell lines such as YO, NSO and Sp2/0. For a review of certain mammalian host cell lines suitable for antibody or antibody derivative production, see, e.g., Yazaki and Wu, Methods in Molecular Biology, Vol. 248 (B. K. C. Lo, ed., Humana Press, Totowa, NJ), pp. 255-268 (2003).

In certain embodiments, techniques for making bispecific and/or multispecific antibodies include, but are not limited to, recombinant expression of two immunoglobulin heavy chain-light chain pairs having the same specificity, where one or two of the heavy chains or the light chains are fuse to an antigen binding moiety (e.g., a single domain antibody, e.g., a VHH) having a different specificity, recombinant coexpression of two immunoglobulin heavy chain-light chain pairs having different specificities (see Milstei n and Cuello, Nature 305: 537 (1983)), PCT Patent Application No. WO 93/08829, and Traunecker et al., EMBO J 10: 3655 (1991)), and “knob-in-hole” engineering (see, e.g., U.S. Pat. No. 5,731,168). Bispecific antibodies can also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science , 229: 81 (1985)); using leucine zippers to produce bi specific antibodies (see, e.g., Kostelny et al., J Immunol., 148(5): 1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J Immunol. 147: 60 (1991).

Bispecific and multispecific molecules of the present disclosure can also be made using chemical techniques (see, e.g., Kranz (1981) Proc. Natl. Acad. Sci. USA 78:5807), “polydoma” techniques (see, e.g., U.S. Pat. No. 4,474,893), or recombinant DNA techniques. Bispecific and multispecific molecules of the presently disclosed subject matter can also be prepared by conjugating the constituent binding specificities, e.g., a first epitope and a second epitope binding specificities, using methods known in the art and as described herein. For example, and not by way of limitation, each binding specificity of the bispecific and multispecific molecule can be generated together by recombinant fusion protein techniques, or can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), N-succinimidyl-3-(2-pyridyldithio)propionate (SPDP), and sulfosuccinimidyl 4-(N-maleimidomethyl) cyclohaxane-1-carboxylate (sulfo-SMCC) (see, e.g., Karpovsky (1984) J. Exp. Med. 160:1686; Liu (1985) Proc. Natl. Acad. Sci. USA 82:8648). Other methods include those described by Paulus (Behring Ins. Mitt. (1985) No. 78, 1 18-132; Brennan (1985) Science 229:81-83), Glennie (1987) J Immunol. 139: 2367-2375). When the binding specificities are antibodies (e.g., two humanized antibodies), they can be conjugated via sulfhydryl bonding of the C-terminus hinge regions of the two heavy chains. In certain embodiments, the hinge region can be modified to contain an odd number of sulfhydryl residues, e.g., one, prior to conjugation.

In certain embodiments, both binding specificities of a bispecific antibody can be encoded in the same vector and expressed and assembled in the same host cell. This method is particularly useful where the bispecific and multispecific molecule is a MAb×MAb, MAb×Fab, Fab×F(ab′)2 or ligand×Fab fusion protein. In certain embodiments, a bispecific antibody of the present disclosure can be a single chain molecule, such as a single chain bispecific antibody, a single chain bispecific molecule comprising one single chain antibody and a binding determinant, or a single chain bispecific molecule comprising two binding determinants. Bispecific and multispecific molecules can also be single chain molecules or can comprise at least two single chain molecules. Methods for preparing bi- and multispecific molecules are described, for example, in U.S. Pat. Nos. 5,260,203; 5,455,030; 4,881,175; 5,132,405; 5,091,513; 5,476,786; 5,013,653; 5,258,498; and 5,482,858. Engineered antibodies with three or more functional antigen binding sites (e.g., epitope binding sites) including “Octopus antibodies”, are also included herein (see, e.g., US 2006/0025576A1).

In certain embodiments, an animal system can be used to produce an antibody or antibody derivative of the present disclosure. One animal system for preparing hybridomas is the murine system.

Hybridoma production in the mouse is a very well-established procedure. Immunization protocols and techniques for isolation of immunized splenocytes for fusion are known in the art. Fusion partners (e.g., murine myeloma cells) and fusion procedures are also known (see, e.g., Harlow and Lane (1988), Antibodies, A Laboratory Manual, Cold Spring Harbor Laboratory Press, Cold Spring Harbor New York).

### 2.10 Assays

The antibodies and antibody derivatives of the present disclosure provided herein can be identified, screened for, or characterized for their physical/chemical properties and/or biological activities by various assays known in the art and provided herein.

In certain embodiments, an antibody or antibody derivative of the present disclosure can be tested for its antigen binding activity by known methods, such enzyme-linked immunosorbent assay (ELISA), a radioimmunoassay (MA), or a Western Blot Assay. Each of these assays generally detects the presence of protein-antibody complexes of particular interest by employing a labeled reagent (e.g., an antibody) specific for the complex of interest. For example, the antibody or antibody derivative can be detected using, e.g., an enzyme-linked antibody or antibody fragment which recognizes and specifically binds to the antibody or antibody derivative. Alternatively, the antibody or antibody derivative can be detected using any of a variety of other immunoassays. For example, the antibody or antibody derivative can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a Geiger counter or a scintillation counter or by autoradiography.

In certain embodiments, competition assays can be used to identify an antibody or antibody derivative that competes with an antibody of the present disclosure for binding to GPC3. In certain embodiments, such a competing antibody binds to the same epitope (e.g., a linear or a conformational epitope) that is bound by an antibody disclosed herein. Detailed exemplary methods for mapping an epitope to which an antibody binds are provided in Morris (1996) “Epitope Mapping Protocols”, in Methods in Molecular Biology vol. 66 (Humana Press, Totowa, NJ).

In a non-limiting example of a competition assay, immobilized GPC3 can be incubated in a solution comprising a first labeled antibody or antibody derivative that binds to GPC3 and a second unlabeled antibody that is being tested for its ability to compete with the first antibody for binding to GPC3. The second antibody may be present in a hybridoma supernatant. As a control, immobilized GPC3 is incubated in a solution comprising the first labeled antibody but not the second unlabeled antibody. After incubation under conditions permissive for binding of the first antibody to GPC3, excess unbound antibody is removed, and the amount of label associated with immobilized GPC3 is measured. If the amount of label associated with immobilized GPC3 is substantially reduced in the test sample relative to the control sample, then that indicates that the second antibody is competing with the first antibody for binding to GPC3. See Harlow and Lane (1988) Antibodies: A Laboratory Manual ch. 14 (Cold Spring Harbor Laboratory, Cold Spring Harbor, NY).

The present disclosure provides assays for identifying anti-GPC3 antibodies or antibody derivatives thereof having biological activity. Biological activity may include, e.g., activating an immune cell or an immune activation reporter, e.g., a NFAT reporter or a NF-κB reporter. Antibodies having such biological activity in vivo and/or in vitro are also provided.

### 2.11 Immunoconjugates

The presently disclosed subject matter further provides immunoconjugates comprising an antibody or antibody derivative, disclosed herein, conjugated to one or more detection probe and/or cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes. For example, an antibody or antigen-binding portion of the disclosed subject matter can be functionally linked (e.g., by chemical coupling, genetic fusion, noncovalent association or otherwise) to one or more other binding molecules, such as another antibody, antibody fragment, peptide or binding mimetic.

In certain embodiments, an immunoconjugate is an antibody drug conjugate (ADC) in which an antibody is conjugated to one or more drugs, including but not limited to a maytansinoid (see U.S. Pat. Nos. 5,208,020, 5,416,064 and European Patent EP 0 425 235); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In certain embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In certain embodiments, an immunoconjugate comprises an antibody as described herein conjugated to a radioactive atom to form a radioconjugate. A variety of radioactive isotopes are available for the production of radioconjugates. Non-limiting examples include At²¹¹, I¹³¹, I¹²⁵, y⁹⁰, Re¹⁸⁶, Re¹⁸⁸, Sm¹⁵³, Bi²¹², P³², Pb²¹² and radioactive isotopes of Lu. When the radioconjugate is used for detection, it can include a radioactive atom for scintigraphic studies, for example tc99m or 1123, or a spin label for nuclear magnetic resonance (NMR) imaging (also known as magnetic resonance imaging, MRI), such as iodine-123, iodine-131, indium-11, fluorine-19, carbon-13, nitrogen-15, oxygen-17, gadolinium, manganese or iron.

Conjugates of an antibody and cytotoxic agent can be made using a variety of bi functional protein coupling agents such as N-succinimid yl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-4-labeled 1-isothiocyanatobenzyl-3-methyl di ethylene triaminepentaacetic acid (MX-DTPA) is an exemplary chelating agent for conjugation of radionucleotide to the antibody. See WO94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-1 31 (1992); U.S. Pat. No. 5,208,020) can be used.

The immunuoconjugates or ADCs herein expressly contemplate, but are not limited to, such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinyl sulfone)benzoate) which are commercially available (e.g., from Pierce Biotechnology, Inc., Rockford, IL., U.S.A).

### 2.12 Antigen-Recognizing Receptor

The presently disclosed subject matter further provides antigen-recognizing receptors comprising an antibody or antibody fragment disclosed herein. An antigen-recognizing receptor is a receptor that is capable of activating, stimulating or inhibiting an immunoresponsive cell (e.g., a T-cell) in response to its binding to an antigen. Non-limiting examples of antigen-recognizing receptors include native and recombinant T cell receptors (“TCRs”), a chimeric co-stimulating receptor (CCR), a chimeric antigen receptors (“CARs”) or an inhibitory CAR (iCAR). Antigen-recognizing receptor designs and methods of use are well known in the art, and is described in the literature, e.g., International Publications WO 2018/027155, WO 2019/099483, WO 2019/157454, WO 2019/133969, WO 2019/099993, WO 2015/142314, WO 2018/027197 and WO 2014055668.

In certain embodiments, the presently disclosed subject matter provides chimeric antigen receptors (CARs) comprising an antibody or antibody fragment disclosed herein. CARs are engineered receptors, which can graft or confer a specificity of interest onto an immune effector cell. In certain embodiments, a CAR can be used to graft the specificity of a monoclonal antibody onto a T cell; with transfer of its coding sequence facilitated by a vector. In certain embodiments, the CAR is a “First generation” CAR, which is typically composed of an extracellular antigen-binding domain (e.g., a scFv or a VHH) fused to a transmembrane domain, which is fused to cytoplasmic/intracellular signaling domain. “First generation” CARs can provide de novo antigen recognition and cause activation of an immunoresponsive cell, e.g., CD4+ and CD8+ T cells, through their CD3z chain signaling domain in a single fusion molecule, independent of HLA-mediated antigen presentation. In certain embodiments, the CAR is a “Second generation” CAR, which further comprises an intracellular signaling domain from various co-stimulatory molecules (e.g., CD28, 4-1BB, ICOS, 0X40, CD27, CD40/My88 and NKGD2) to the cytoplasmic tail of the CAR to provide additional signals to the immunoresponsive cell, whereby the “Second generation” CAR comprise those that provide both co-stimulation (e.g., CD28 or 4-1BB) and activation (CD3z). In certain embodiments, the CAR is a “Third generation” CAR, which comprises multiple co-stimulation domains (e.g., CD28 and 4-1BB) and activation (CD3z). In certain embodiments, the CAR is a second-generation CAR. In certain embodiments, the CAR comprises an extracellular antigen-binding domain that binds to an antigen, a transmembrane domain, and an intracellular signaling domain, wherein the intracellular signaling domain comprises a co-stimulatory signaling domain. In certain embodiments, the CAR further comprises a hinger/spacer region between the extracellular antigen-binding domain and the transmembrane domain. In certain embodiments, the extracellular antigen-binding domain comprises an antibody or antibody fragment disclosed herein. In certain embodiments, the antibody or antibody fragment comprises a VHH or a scFv.

In certain embodiments, the presently disclosed subject matter provides recombinant TCRs comprising an antibody or antibody fragment disclosed herein. A native TCR is a protein complex comprising a disulfide-linked heterodimeric protein consisting of two variable chains expressed as part of a complex with CD3 chain molecules. A native TCR is found on the surface of T cells, and is responsible for recognizing antigens as peptides bound to major histocompatibility complex (MHC) molecules. In certain embodiments, a native TCR comprises an alpha chain and a beta chain (encoded by TRA and TRB genes, respectively). In certain embodiments, a TCR comprises a gamma chain and a delta chain (encoded by TRG and TRD genes, respectively). Each of the alpha chain, the beta chain, the gamma chain and the delta chain is comprises two extracellular domains: a Variable (V) region and a Constant (C) region. The Constant region is proximal to the cell membrane, followed by a transmembrane region and a short cytoplasmic tail. The Variable region binds to the peptide/MHC complex. Each variable region has three complementarity determining regions (CDRs). In certain embodiments, a TCR comprises a receptor complex with CD3δ, CD3γ, CD3ε and CD3ζ. When a TCR complex engages with its antigen and MHC (peptide/MHC), the T cell expressing the TCR complex is activated.

In certain embodiments, a recombinant TCR is a non-naturally occurring TCR. In certain embodiments, the recombinant TCR comprises a recombinant alpha chain and/or a recombinant b chain, wherein a part or the entire variable region of the recombinant alpha chain and/or the recombinant b chain is replaced by an antibody or an antibody fragment disclosed herein. In certain embodiments, the antibody or antibody fragment comprises a VHH, a VH, a VL or a scFv. In certain embodiments, the antibody or antibody fragment comprises a VHH. In certain embodiments, the recombinant TCR binds to an antigen of interest in an MHC/HLA-independent manner. In certain non-limiting embodiments, binding of the antigen is capable of activating an immunoresponsive cell comprising the recombinant TCR.

The presently disclosed subject matter provides immunoresponsive cells comprising (a) an antigen-recognizing receptor (e.g., CAR or TCR) disclosed herein. In certain embodiments, the antigen-recognizing receptor is capable of activating the immunoresponsive cell. The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g, TEM cells and TEMRA cells, Regulatory T cells (also known as suppressor T cells), Natural killer T cells, Mucosal associated invariant T cells, and gd T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen recognizing receptor, e.g., a CAR or a TCR. In certain embodiments, the immunoresponsive cell is a T cell. The T cell can be a CD4+ T cell or a CD8+ T cell. In certain embodiments, the T cell is a CD4+ T cell. In certain embodiments, the T cell is a CD8+ T cell. Natural killer (NK) cells can be lymphocytes that are part of cell-mediated immunity and act during the innate immune response. NK cells do not require prior activation in order to perform their cytotoxic effect on target cells. Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314: 126-129 (disclosing peripheral donor lymphocytes genetically modified to express a full-length tumor antigen-recognizing T cell receptor complex comprising the a and b heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). In certain embodiments, the immunoresponsive cells (e.g., T cells) can be autologous, non-autologous (e.g., allogeneic), or derived in vitro from engineered progenitor or stem cells.

### 3. Methods of Use

The presently disclosed subject matter further provides methods for using the disclosed antibodies and antibody derivatives. In certain embodiments, the methods are directed to therapeutic uses of a presently disclosed antibody or antibody derivative. In certain embodiments, the methods are directed to diagnostic use of a presently disclosed antibody or antibody derivative.

### 3.1 Treatment Methods

The present disclosure provides methods and use of an antibody or antibody derivative disclosed herein for treatment of diseases and disorders or for increasing an immune response. In certain embodiments, the antibody, antibody derivative or pharmaceutical compositions comprising the same disclosed herein can be administered to subjects (e.g., mammals such as humans) to treat diseases and disorders or to increases an immune response. In certain embodiments, the diseases and disorders involve immune checkpoint inhibitions and/or abnormal GPC3 activity. In certain embodiments, the diseases and disorders that can be treated by an antibody or antibody derivative disclosed herein include, but are not limited to, neoplasia, e.g., cancer.

In certain embodiments, the present disclosure provides an antibody or antibody derivative described herein (or fragments thereof) for use in the manufacture of a medicament. In certain embodiments, the present disclosure provides antibody or antibody derivative described herein (or fragments thereof) for use in the manufacture of a medicament for treating of cancer. In certain embodiments, the present disclosure provides an antibody or antibody derivative described herein (or fragments thereof) for use in treating cancer in a subject. In certain embodiments, the present disclosure provides pharmaceutical compositions comprising an antibody or antibody derivative provided herein (or fragments thereof) for use in treating cancer in a subject. In certain embodiments, the cancer can be blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known carcinoma in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas.

In certain embodiments, the cancer can be melanoma, NSCLC, head and neck cancer, urothelial cancer, breast cancer (e.g., triple-negative breast cancer, TNBC), gastric cancer, cholangiocarcinoma, classical Hodgkin's lymphoma (cHL), Non-Hodgkin lymphoma primary mediastinal B-Cell lymphoma (NHL PMBCL), mesothelioma, ovarian cancer, lung cancer (e.g., small-cell lung cancer), esophageal cancer, nasopharyngeal carcinoma (NPC), biliary tract cancer, colorectal cancer, cervical cancer or thyroid cancer.

In certain embodiments, the subject to be treated is a mammal (e.g., human, non-human primate, rat, mouse, cow, horse, pig, sheep, goat, dog, cat, etc.). In certain embodiments, the subject is a human. In certain embodiments, the subject is suspected of having or at risk of having a cancer or be diagnosed with a cancer or any other disease having abnormal GPC3 expression or activity.

Many diagnostic methods for cancer or any other disease exhibiting abnormal GPC3 activity and the clinical delineation of those diseases are known in the art. Such methods include, but are not limited to, e.g., immunohistochemistry, PCR, fluorescent in situ hybridization (FISH). Additional details regarding diagnostic methods for abnormal GPC3 activity or expression are described in, e.g., Gupta et al. (2009) Mod Pathol. 22(1): 128-133; Lopez-Rios et al. (2013) J Clin Pathol. 66(5): 381-385; Ellison et al. (2013) J Clin Pathol 66(2): 79-89; and Guha et al. (2013) PLoS ONE 8(6): e67782.

Administration can be by any suitable route including, e.g., intravenous, intramuscular, or subcutaneous. In some embodiments, the antibody or antibody derivative (or fragments thereof) and/or compositions provided herein are administered in combination with a second, third, or fourth agent (including, e.g., an antineoplastic agent, a growth inhibitory agent, a cytotoxic agent, or a chemotherapeutic agent) to treat the diseases or disorders involving abnormal GPC3 activity. Such agents include, e.g., docetaxel, gefitinib, FOLFIRI (irinotecan, 5-fluorouracil, and leucovorin), irinotecan, cisplatin, carboplatin, paclitaxel, bevacizumab (anti-VEGF antibody), FOLFOX-4, infusional fluorouracil, leucovorin, and oxaliplatin, afatinib, gemcitabine, capecitabine, pemetrexed, tivantinib, everolimus, CpG-ODN, rapamycin, lenalidomide, vemurafenib, endostatin, lapatinib, PX-866, Imprime PGG, and irlotinibm. In some embodiments, the antibody or antibody derivative (or fragments thereof) is conjugated to the additional agent.

In certain embodiments, the antibody or antibody derivative (or fragments thereof) and/or compositions provided herein are administered in combination with one or more additional therapies, such as radiation therapy, surgery, chemotherapy, and/or targeted therapy. In certain embodiments, the antibody, antibody derivative (or fragments thereof) and/or compositions provided herein are administered in combination with radiation therapy. In certain embodiments, the combination of an antibody, antibody derivative (or fragment thereof) and/or composition provided herein and radiation therapy is used for treating a neoplasm or cancer disclosed herein.

Depending on the indication to be treated and factors relevant to the dosing that a physician of skill in the field would be familiar with, the antibody or antibody derivative provided herein will be administered at a dosage that is efficacious for the treatment of that indication while minimizing toxicity and side effects. For the treatment of a cancer, a typical dose can be, for example, in the rage of 0.001 to 1000 μg; however, doses below or above this exemplary range are within the scope of the invention. The daily dose can be about 0.1 μg/kg to about 100 mg/kg of total body weight, about 0.1 μg/kg to about 100 μg/kg of total body weight or about 1 μg/kg to about 100 μg/kg of total body weight. As noted above, therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

A pharmaceutical composition comprising an antibody or antibody derivative disclosed herein can be administered one, two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection or surgically implanted in various locations.

Cancer treatments can be evaluated by, e.g., but not limited to, tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In certain embodiments, the efficacy of treatment is measured by the percentage tumor growth inhibition (% TGI), calculated using the equation 100−(T/C×100), where T is the mean relative tumor volume of the treated tumor, and C is the mean relative tumor volume of a non-treated tumor. In certain embodiments, the % TGI is about 10%, about 20%, about 30%, about 40%, about 50%, about 60%, about 70%, about 80%, about 90%, about 91%, about 92%, about 93%), about 94%), about 95%, or more than 95%.

### 3.2 Methods of Diagnosis and Imaging

Labeled antibody or antibody derivative can be used for diagnostic purposes to detect, diagnose, or monitor diseases and/or disorders associated with the expression, aberrant expression and/or activity of GPC3. For example, the antibodies and antibody derivatives provided herein can be used in in situ, in vivo, ex vivo, and in vitro diagnostic assays or imaging assays. Methods for detecting expression of a GPC3 polypeptide, comprising (a) assaying the expression of the polypeptide in cells (e.g., tissue) or body fluid of an individual using one or more antibody or antibody derivative and (b) comparing the level of gene expression with a standard gene expression level, whereby an increase or decrease in the assayed gene expression level compared to the standard expression level is indicative of aberrant expression.

Additional embodiments provided herein include methods of diagnosing a disease or disorder associated with expression or aberrant expression of GPC3 in an animal (e.g., a mammal such as a human). The methods comprise detecting GPC3 molecules in the mammal. In certain embodiments, diagnosis comprises: (a) administering an effective amount of a labeled antibody or antibody derivative to a mammal (b) waiting for a time interval following the administering for permitting the labeled antibody or antibody derivative to preferentially concentrate at sites in the subject where the GPC3 molecule is expressed (and for unbound labeled molecule to be cleared to background level); (c) determining background level; and (d) detecting the labeled molecule in the subject, such that detection of labeled molecule above the background level indicates that the subject has a particular disease or disorder associated with expression or aberrant expression of GPC3. Background level can be determined by various methods including, comparing the amount of labeled molecule detected to a standard value previously determined for a particular system.

Antibodies and antibody derivatives provided herein can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹³¹I, ¹²⁵I, ¹²³I, ¹²¹I), carbon (¹⁴C), sulfur (³⁵S), tritium (³H), indium (¹¹⁵In, ^{113m}In, ¹¹²In, ¹¹¹In), and technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru; luminol; and fluorescent labels, such as fluorescein and rhodamine, and biotin.

Techniques known in the art may be applied to labeled antibodies (or fragments thereof) provided herein. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003).

Alternatively, or additionally, one can measure levels of a GPC3 polypeptide-encoding nucleic acid or mRNA in the cell, e.g., via fluorescent in situ hybridization using a nucleic acid based probe corresponding to a GPC3-encoding nucleic acid or the complement thereof; (FISH; see WO98/45479 published October, 1998), Southern blotting, Northern blotting, or polymerase chain reaction (PCR) techniques, such as real time quantitative PCR (RT-PCR). One can also study GPC3 overexpression by measuring shed antigen in a biological fluid such as serum, e.g., using antibody-based assays (see also, e.g., U.S. Pat. No. 4,933,294 issued Jun. 12, 1990; WO91/05264 published Apr. 18, 1991; U.S. Pat. 5,401,638 issued Marc. 28, 1995; and Sias et al., J. Immunol. Methods 132:73-80 (1990)). Aside from the above assays, various in vivo and ex vivo assays are available to the skilled practitioner. For example, one can expose cells within the body of the mammal to an antibody which is optionally labeled with a detectable label, e.g., a radioactive isotope, and binding of the antibody to the body cells can be evaluated, e.g., by external scanning for radioactivity or by analyzing a sample (e.g., a biopsy or other biological sample) taken from a mammal previously exposed to the antibody.

### 4. Pharmaceutical Formulations

The presently disclosed subject matter further provides pharmaceutical formulations containing an antibody or antibody derivative disclosed herein, with a pharmaceutically acceptable carrier. In certain embodiments, the pharmaceutical compositions can include a combination of multiple (e.g., two or more) antibodies and/or antibody derivatives of the presently disclosed subject matter.

In certain embodiments, the disclosed pharmaceutical formulations can be prepared by combining an antibody or antibody derivative having the desired degree of purity with one or more optional pharmaceutically acceptable carriers (Remington's Pharmaceutical Sciences 16th edition, Osol, A. Ed. (1980)), in the form of lyophilized formulations or aqueous solutions. For example, but not by way of limitation, lyophilized antibody formulations are described in U.S. Pat. No. 6,267,958. In certain embodiments, aqueous antibody formulations can include those described in U.S. Pat. No. 6,171,586 and WO2006/044908, the latter formulations including a histidine-acetate buffer. In certain embodiments, the antibody or antibody derivative can be of a purity greater than about 80%, greater than about 90%, greater than about 91%, greater than about 92%, greater than about 93%, greater than about 94%, greater than about 95%, greater than about 96%, greater than about 97%, greater than about 98%, greater than about 99%, greater than about 99.1%, greater than about 99.2%, greater than about 99.3%, greater than about 99.4%, greater than about 99.5%, greater than about 99.6%, greater than about 99.7%, greater than about 99.8% or greater than about 99.9%.

Pharmaceutically acceptable carriers are generally nontoxic to recipients at the dosages and concentrations employed, and include, but are not limited to: buffers such as phosphate, citrate, and other organic acids, antioxidants including ascorbic acid and methionine, preservatives (such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight (less than about 10 residues) polypeptides, proteins, such as serum albumin, gelatin, or immunoglobulins, hydrophilic polymers such as polyvinylpyrrolidone, amino acids such as glycine, glutamine, asparagine, histidine, arginine, or lysine, monosaccharides, disaccharides, and other carbohydrates including glucose, mannose, or dextrins, chelating agents such as EDTA, sugars such as sucrose, mannitol, trehalose or sorbitol, salt-forming counter-ions such as sodium, metal complexes (e.g., Zn-protein complexes), and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In certain embodiments, a sHASEGP is combined with one or more additional glycosaminoglycanases such as chondroitinases.

The carrier can be suitable for intravenous, intramuscular, subcutaneous, parenteral, spinal or epidermal administration (e.g., by injection or infusion). Depending on the route of administration, the active compound, e.g., an anti-GPC3 antibody, can be coated in a material to protect the compound from the action of acids and other natural conditions that may inactivate the compound.

Pharmaceutical compositions of the present disclosure also can be administered in combination therapy, i.e., combined with other agents. In certain embodiments, pharmaceutical compositions disclosed herein can also contain more than one active ingredients as necessary for the particular indication being treated, for example, those with complementary activities that do not adversely affect each other. In certain embodiments, the pharmaceutical formulation can include a second active ingredient for treating the same disease treated by the first therapeutic. Such active ingredients are suitably present in combination in amounts that are effective for the purpose intended. For example, and not by way of limitation, the formulation of the present disclosure can also contain more than one active ingredients as necessary for the particular indication being treated, preferably those with complementary activities that do not adversely affect each other. For example, it may be desirable to further provide a second therapeutic useful for treatment of the same disease. Such active ingredients are suitably present in combination in amounts that are effective for the purpose intended.

A composition of the present disclosure can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, 1978. In certain embodiments, the pharmaceutical compositions are manufactured under Good Manufacturing Practice (GMP) conditions of the U.S. Food and Drug Administration.

Sustained-release preparations containing an antibody or antibody derivative disclosed herein can also be prepared. Suitable examples of sustained-release preparations include semipermeable matrices of solid hydrophobic polymers containing the antibody or antibody derivative, which matrices are in the form of shaped articles, e.g. films, or microcapsules. In certain embodiments, active ingredients can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

To administer an antibody or antibody derivative of the present disclosure by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art.

Except insofar as any conventional media or agent is incompatible with the active compound, use thereof in the pharmaceutical compositions of the present disclosure is contemplated. Supplementary active compounds can also be incorporated into the compositions.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating one or more antibody or antibody derivative disclosed herein in the required amount in an appropriate solvent with one or a combination of ingredients enumerated above, as required, followed by sterilization microfiltration, e.g., by filtration through sterile filtration membranes. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Therapeutic compositions can also be administered with medical devices known in the art. For example, a therapeutic composition of the present disclosure can be administered with a needleless hypodermic injection device, such as the devices disclosed in, e.g., U.S. Pat. Nos. 5,399,163, 5,383,851, 5,312,335, 5,064,413, 4,941,880, 4,790,824 or 4,596,556. Examples of implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

For the therapeutic compositions, formulations of the present disclosure include those suitable for oral, nasal, topical (including buccal and sublingual), rectal, vaginal and/or parenteral administration. The formulations can conveniently be presented in unit dosage form and may be prepared by any methods known in the art of pharmacy. The amount of antibody or antibody derivative, which can be combined with a carrier material to produce a single dosage form, vary depending upon the subject being treated, and the particular mode of administration. The amount of the antibody or antibody derivative which can be combined with a carrier material to produce a single dosage form generally be that amount of the composition which produces a therapeutic effect. Generally, out of one hundred percent, this amount range from about 0.01 percent to about ninety-nine percent of active ingredient, from about 0.1 percent to about 70 percent, or from about 1 percent to about 30 per cent.

Dosage forms for the topical or transdermal administration of compositions of the present disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

These pharmaceutical compositions can also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

In certain embodiments, when an antibody or antibody derivative of the present disclosure are administered as pharmaceuticals, to humans and animals, they can be given alone or as a pharmaceutical composition containing, for example, from about 0.01% to about 99.5% (or about 0.1% to about 90%) of the antibody or antibody derivative in combination with a pharmaceutically acceptable carrier.

### 5. Articles of Manufacture

The presently disclosed subject matter further provides articles of manufacture containing materials useful for the treatment, prevention and/or diagnosis of the disorders described above.

In certain embodiments, the article of manufacture includes a container and a label or package insert on or associated with the container. Non limiting examples of suitable containers include bottles, vials, syringes, IV solution bags, etc. The containers can be formed from a variety of materials such as glass or plastic. The container can hold a composition which is by itself or combined with another composition effective for treating, preventing and/or diagnosing the condition and may have a sterile access port (for example, the container may be an intravenous solution bag or a vial having a stopper pierceable by a hypodermic injection needle).

In certain embodiments, at least one active agent in the composition is an antibody or antibody derivative of the present disclosure. The label or package insert can indicate that the composition is used for treating the condition of choice.

In certain embodiments, the article of manufacture can comprise (a) a first container with a composition contained therein, wherein the composition comprises an antibody or antibody derivative of the present disclosure; and (b) a second container with a composition contained therein, wherein the composition comprises a further cytotoxic or otherwise therapeutic agent. In certain embodiments, the article of manufacture can further comprise a package insert indicating that the compositions can be used to treat a particular condition.

Alternatively, or additionally, the article of manufacture can further an additional container, e.g., a second or third container, including a pharmaceutically acceptable buffer, such as, but not limited to, bacteriostatic water for injection (BWFI), phosphate-buffered saline, Ringer's solution and dextrose solution. The article of manufacture can include other materials desirable from a commercial and user standpoint, including other buffers, diluents, filters, needles, and syringes.

SEQUENCE TABLE

| SEQ ID NO | GENE NAME | AMINO ACID SEQUENCE |
|---|---|---|
| 1. | 4F3 llama | GFTFSSYI |
| | CDR1 | |
| 2. | 4F3 llama | ISTGGKST |
| | CDR2 | |
| 3. | 4F3 llama | AKGGKSRSYYSE |
| | CDR3 | |
| 4. | 4F3 llama | QVQLQESGGGLVQPGGSLRLSCAASGFTFSSYIMSWIRQA |
| | VHH | PGKELEWVATISTGGKSTAYADSVKGRFTVSRDNAINTAY |
| | | LQMNSLKSEDTAVYYCAKGGKSRSYYSERGQGTLVTVSS |
| 5. | 1B01 llama | GLPFSNYA |
| | CDR1 | |
| 6. | 1B01 llama | VSANGGNE |
| | CDR2 | |
| 7. | 1B01 llama | ATVRRRGGTFTVGSY |
| | CDR3 | |
| 8. | 1B01 llama | QVQLQESGGGLVQAGGSLRLSCAAVGLPFSNYAMGWFRQA |
| | VHH | PGEEREFVSAVSANGGNEYYADSVKDRFTISRDNAKNTVY |
| | | LRMLSLKLEDTAIYYCATVRRRGGTFTVGSYRGQGTQVTV |
| | | SS |
| 9. | 1B01 CDR1 | GLPFSNYA |
| 10. | 1B01 CDR2 | VSANGGNE |
| 11. | 1B01 CDR3 | ATVRRRGGTFTVGSY |
| 12. | 1B01 VHH | QVQLVESGGGLVQPGGSLRLSCAAVGLPFSNYAMGWFRQA |
| | | PGKGLEFVSAVSANGGNEYYADSVKGRFTISRDNSKNTLY |
| | | LQMNSLRAEDTAVYYCATVRRRGGTFTVGSYRGQGTQVTV |
| | | SS |
| 13. | 1B01 VHH | QVQLVESGGGLVQPGGSLRLSCAAVGLPFSNYAMGWFRQA |
| | Fc | PGKGLEFVSAVSANGGNEYYADSVKGRFTISRDNSKNTLY |
| | | LQMNSLRAEDTAVYYCATVRRRGGTFTVGSYRGQGTQVTV |
| | | SSEPKSCDKTHTCPPCPAPELLGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPAPIE |
| | | KTISKAKGQPREPQVYTLPPSREEMTKNQVSLTCLVKGFY |
| | | PSDIAVEWESNGQPENNYKTTPPVLDSDGSFFLYSKLTVD |
| | | KSRWQQGNVFSCSVMHEALHNHYTQKSLSLSPGK |
| 14. | Human | MAGTVRTACLVVAMLLSLDFPGQAQPPPPPPDATCHQVRS |
| | GPC3 | FFQRLQPGLKWVPETPVPGSDLQVCLPKGPTCCSRKMEEK |
| | polypeptide | YQLTARLNMEQLLQSASMELKFLIIQNAAVFQEAFEIVVR |
| | | HAKNYTNAMFKNNYPSLTPQAFEFVGEFFTDVSLYILGSD |
| | | INVDDMVNELFDSLFPVIYTQLMNPGLPDSALDINECLRG |
| | | ARRDLKVFGNFPKLIMTQVSKSLQVTRIFLQALNLGIEVI |
| | | NTTDHLKFSKDCGRMLTRMWYCSYCQGLMMVKPCGGYCNV |
| | | VMQGCMAGVVEIDKYWREYILSLEELVNGMYRIYDMENVL |
| | | LGLFSTIHDSIQYVQKNAGKLTTTETEKKIWHFKYPIFFL |
| | | CIGLDLQIGKLCAHSQQRQYRSAYYPEDLFIDKKVLKVAH |
| | | VEHEETLSSRRRELIQKLKSFISFYSALPGYICSHSPVAE |
| | | NDTLCWNGQELVERYSQKAARNGMKNQFNLHELKMKGPEP |
| | | VVSQIIDKLKHINQLLRTMSMPKGRVLDKNLDEEGFESGD |
| | | CGDDEDECIGGSGDGMIKVKNQLRFLAELAYDLDVDDAPG |
| | | NSQQATPKDNEISTFHNLGNVHSPLKLLTSMAISVVCFFF |
| | | LVH |
| 15. | C-terminal | SAYYPEDLFIDKKVLKVAHVEHEETLSSRRRELIQKLKSF |
| | ECD of | ISFYSALPGYICSHSPVAENDTLCWNGQELVERYSQKAAR |
| | Human | NGMKNQFNLHELKMKGPEPVVSQIIDKLKHINQLLRTMSM |
| | GPC3 | PKGRVLDKNLDEEGFESGDCGDDEDECIGGSGDGMIKVKN |
| | polypeptide | QLRFLAELAYDLDVDDAPGNSQQATPKDNEISTFHN |
| 16. | Exemplary | GGGGSGGGGS |
| | linker | |
| 17. | Exemplary | GSGGSGGSGGSG |
| | linker | |
| 18. | Exemplary | GGGGSGGGGSGGGGS |
| | linker | |
| 19. | Exemplary | GGGSG |
| | linker | |
| 20. | Exemplary | GGGSGGGGSG |
| | linker | |
| 21. | Exemplary | GGSGGGSG |
| | linker | |
| 22. | Exemplary | GGSGGGSGGGSG |
| | linker | |
| 23. | Exemplary | GSGGSG |
| | linker | |
| 24. | Exemplary | GSGGSGGSG |
| | linker | |
| 25. | Exemplary | GSGSGSG |
| | linker | |
| 26. | Exemplary | GSGGSGGSGGSG |
| | linker | |
| 27. | Exemplary | GGGGSGGGGSGGGGSGGG |
| | linker | |
| 28. | Exemplary | GGGGSGGGGSGGGGSGGGGSGGGGS |
| | linker | |
| 29. | Exemplary | GGGGSGGGGSGGGGSGGGGSGGGGSGGGGS |
| | linker | |
| 30. | Exemplary | GGGGSGGGGSGGGGSGGGGS |
| | linker | |
| 31. | Exemplary | PAPAP |
| | linker | |
| 32. | Exemplary | PAPAPPAPAPPAPAP |
| | linker | |
| 33. | Exemplary | IKRTVAA |
| | linker | |
| 34. | Exemplary | VSSASTK |
| | linker | |
| 35. | Exemplary | GGGGSGASTK |
| | linker | |
| 36. | Exemplary | ASTKGGGGSG |
| | linker | |
| 37. | Exemplary | ASTK |
| | linker | |
| 38. | Exemplary | ASTKSGGSGGSG |
| | linker | |
| 39. | Exemplary | AEAAAKA |
| | linker | |
| 40. | Exemplary | AEAAAKEAAAKA |
| | linker | |
| 41. | Exemplary | GRPGS GRPGS |
| | linker | |
| 42. | Exemplary | GRPGS GRPGS GRPGS GRPGS |
| | linker | |
| 43. | Exemplary | GRGGS GRGGS |
| | linker | |
| 44. | Exemplary | GRGGS GRGGS GRGGS GRGGS |
| | linker | |
| 45. | Exemplary | GKPGS GKPGS |
| | linker | |
| 46. | Exemplary | GKPGS GKPGS GKPGS GKPGS |
| | linker | |
| 47. | Exemplary | GEPGS GEPGS |
| | linker | |
| 48. | Exemplary | GEGGS GEGGS GEGGS GEGGS |
| | linker | |
| 49. | Exemplary | GDPGS GDPGS |
| | linker | |
| 50. | Exemplary | GDPGS GDPGS GDPGS GDPGS |
| | linker | |

The following examples are merely illustrative of the presently disclosed subject matter and should not be considered as limitations in any way.

### EXAMPLES

### Example 1. Generation and Screening of Anti-GPC3 VHH Antibodies

Antigen of recombinant human GPC3 extra cellular domain (ECD) protein was constructed with either C-terminal poly-histidine Tag or Fc tag and purified in house. Immunization of llama using GPC3 ECD-His was performed using a mix of 1 mg immunogen and CFA/IFA adjuvants in a final volume of 2 ml. The titer of serum and the presence of GPC3-specific antibodies was confirmed by ELISA using the sera obtained from test bleeds at pre-immune and 52 days' time points. Whole blood was then collected, and PBMCs were isolated. Total RNA was then isolated from the PBMCs, and the cDNA of the antibody V region was synthesized from the total RNA using SuperScript IV Reverse Transcriptase First-strand cDNA Synthesis Kit (Thermo Fisher #18091050). The variable regions of the VH and the VHH antibody genes were amplified by PCR using standard protocols from the cDNA using forward primers annealing to the V segment and reverse primers annealing in the CH2 regions of llama antibody isotypes of IgG1, 2 and 3. The VHH gene from IgG2 and IgG3 llama antibodies was then isolated by gel extraction. Secondary nested PCRs were performed to amplify the V segment of gel purified VHH gene and to add restriction enzyme sites 5′ and 3′ for cloning into a phagemid vector pADL-23c (Antibody Design Labs). Ligated DNA was transformed into electrocompetent TG1 (Lucigen) cells (1.2 μg DNA in 60 μL TG1 cells). Transformations were repeated for 10 times to reach 10⁸˜10⁹ library size. Transformations were spread on 2×YT medium with 2% glucose and 100 μg/mL carbenicillin, which were incubated overnight at 30° C. The following morning the bacteria were scraped from the plates, combined, and stored in 15% glycerol 2×YT at —80° C.

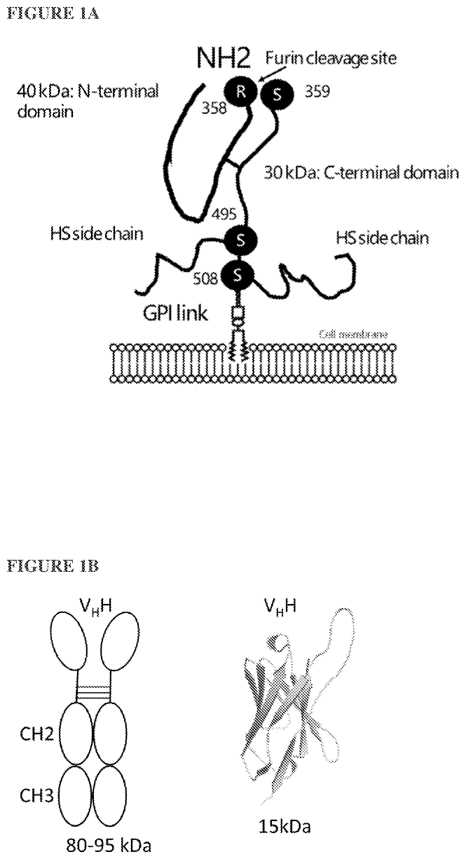

To identify anti-GPC3 specific VHH antibodies, transformed TG1 cells were cultured in 2×YT medium in the presence of helper phage and incubated overnight. The phages in supernatants of cell culture were harvested by centrifugation, and panning for binders to human GPC3 antigen was performed using solution phase panning as previously described (Hawkins et al., J. Mol. Biol., 226 (1992), p. 889; Vaughan et al., Nat. Biotechnol., 14 (1996), p. 309). Prior to panning, GPC3-His was biotinylated with the EZ-Link Sulfo-NHS-LC-Biotin, No-Weigh Format (Thermo Fisher #A39258). Biotinylated human GPC3 was then coated into streptavidin-coupled Dynabeads (Thermo Fisher #11206D). After one round of panning, binders of GPC3 were eluted, which were used to infect SS320 cells. Colonies of the SS320 cells were picked and cultured in Y2T medium, and IPTG was added for secretion of VHH antibodies. Supernatants with VHH antibodies were screened by ELISA assays using recombinant human GPC3 coated plates. Positive human GPC3 binders were picked for sequencing. Clones with different sequences were selected for additional ELISA screening on recombinant human C-terminal GPC3 and cynomolgus GPC3 proteins. Binders to the C-terminal ECD were preferred over the N-terminal ECD, as the C-terminal ECD is better attached to cell membrane, whereas the N-terminal ECD is more likely to be cleaved and removed from cell surface (FIG. 1A).

ELISA assays using plate bound GPC3 proteins was performed using standard methods. Briefly, 96-well ELISA plates (Costar High Binding) were coated by incubating with 1 μg/mL of either recombinant human GPC3-Fc, GPC3 His, GPC3 C-terminal (LSBIO # LS-G13157-10) or cynomolgus GPC3-His (R&D Systems) in PBS overnight at room temperature. Plates were then washed four times with wash buffer PBST (PBS, 0.05% Tween-20) and blocked with 5% milk in PBS for 1 hour at room temperature. After the blocking solution was discarded, bacterial supernatant was added and incubated for 30 min at room temperature (RT). The wells were washed 3 times with PBST and then incubated with anti-c-Myc horseradish peroxidase (HRP) (Jackson Immuno Research, 1:10,000 dilution) at RT for 30 minutes. For phage ELISA, anti-M13 mAb horseradish peroxidase (HRP) conjugate (Amersham Pharmacia) diluted 1:1000 in PBST was added for 30 min at RT. After being washed 5 times, 50 μl of 3,3′,5,5′-tetramethylbenzidine solution (1-Step™ Turbo TMB, Pierce) was added into each well, incubated for 10 minutes at room temperature, and the reaction was stopped by adding 50 μl of 1 M sulfuric acid. The absorbance at 450 nm was measured on a microplate reader. Two top clones, 1B01 and 4F2, were selected for further testing.

To evaluate antigen binding in vitro, VHH bivalent chimeric antibodies were designed. The human IgG1 Fc was fused to the C-terminal of each VHH to form the llama-human chimeric bivalent (VHH-Fc, FIG. 1B). The resulting constructs were expressed in Expi-CHO transient system and purified in house. Briefly, the cell culture medium was clarified by centrifugation followed by sterile filtration using a 0.2 um filter. Clarified harvest was purified using protein A affinity chromatography, such as GE's Mabselect. The eluted protein was neutralized with 1 M Tris pH 8.5 to pH 5.5. A reference anti-GPC3 VHH antibody, HN3, was developed by the National Institute of Health and disclosed in International Publication No. WO2012145469A1. A bivalent VHH-Fc form of HN3 was made in-house as a control anti-GPC3 VHH antibody.

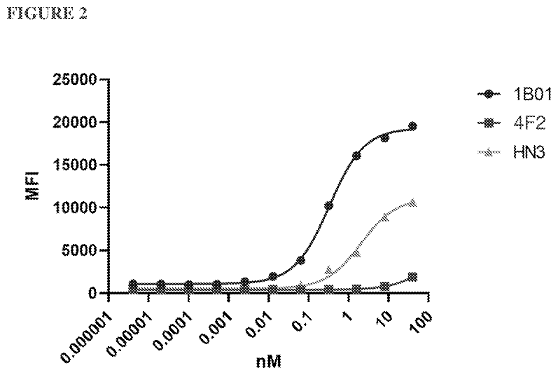

FIG. 2 depicts the binding ability of the two top VHH-Fc clones to HepG2 hepatoma cell line by flow cytometry. HepG2 hepatoma cells express GPC3 endogenously. HN3 VHH-Fc was used as a positive control. Clone 1B01 showed the best binding activity to HepG2 hepatoma cell lines and thus was selected for humanization and further characterization.

### Example 2. Generation of Humanized Anti-GPC3 VHH-Fc Bivalent Antibodies

VHH genes are highly homologous to the human VH3 family of clan III with the exception of several key amino acid substitutions in FR2, namely, Val37→Phe/Tyr, Gly44→Glu, Leu45→Arg, and Trp47→Gly (Kabat numbering). To humanize 1B01 antibody, the “non-human” residues in the framework 1, 2, 3 and 4 were replaced with the closest human VH sequence except for 2 key residues above. 1B01 variable region was subjected to homology search from publicly disclosed IgGblast, Abysis and IMGT databases. As a result, IGHV3-64*04 was utilized. The humanized 1B01 VHH-Fc construct was cloned into expression vector (AS-puro from EMD Millipore), and antibody protein was produced by transient transfection of ExpiCHO and purified by protein A. The HN3 analog described above was used as a positive control.

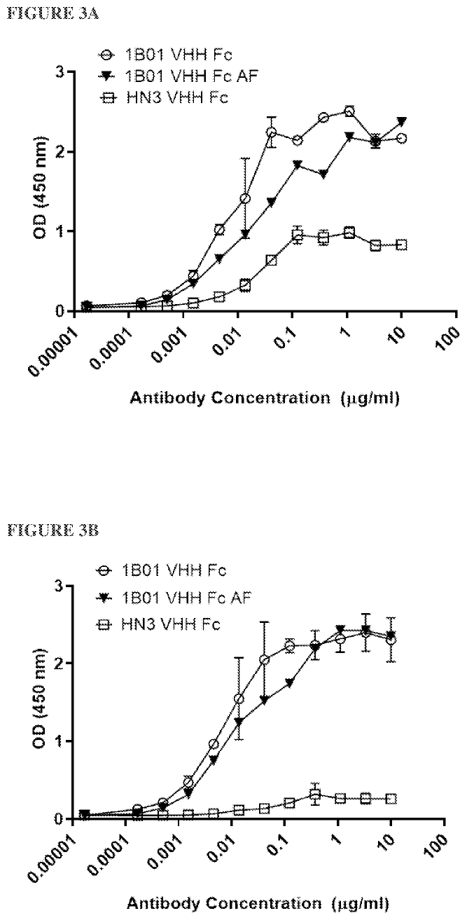

The binding ability of 1B01 VHH-Fc to various versions of GPC3 protein is shown in FIGS. 3A-3D. First, 1B01 VHH-Fc and its afucosylated version (AF) showed stronger binding to human GPC3 protein compared to the HN3 analog (FIG. 3A). Furthermore, 1B01 VHH-Fc and its afucosylated version (AF) both bound to cynomolgus monkey GPC3 protein, whereas the HN3 analog did not show any binding to cynomolgus monkey GPC3 protein (FIG. 3B). In contrast, HN3 bound to mouse GPC3 protein, whereas neither 1B01 VHH-Fc nor its afucosylated version (AF) showed any binding to mouse GPC3 protein (FIG. 3C). Moreover, 1B01 VHH-Fc and its afucosylated version (AF) showed strong binding to human C-terminal ECD of GPC3, whereas HN3 showed no binding to the C-terminal ECD of GPC3 (FIG. 3D), suggesting that 1B01 and HN3 bind to different regions of GPC3. This result is consistent with previous report showing that HN3 binds to a conformational epitope formed by both the N-terminal and C-terminal domains of GPC3 (see WO2012145469A1). As the N-terminal domain can be cleaved and removed from cell surface in certain physiological conditions, antibodies like 1B01 that do not rely on N-terminal domain for antigen-binding can have better tumor targeting ability compared to antibodies like HN3 that require the N-terminal domain for antigen-binding.

### Example 3. In Vitro Characterization of Anti-GPC3 Antibody 1B01

To evaluate the binding activity of the anti-GPC3 antibody 1B01, concentration-dependent binding of the antibody was measured by flow cytometry against HepG2 and Hep3 human hepatoma cell lines (purchased from ATCC), each of which expresses GPC3 endogenously. The cells were grown as a monolayer in Eagle's Minimum Essential Medium (EMEM) supplemented with 10% FBS. Cells were rinsed with 1×PBS (Gibco) twice and incubated with pre-warmed (37° C.) 0.05% Trypsin-EDTA solution for 5-7 minutes. As cells detach, trypsin was neutralized by adding 4× volume of complete growth medium with 10% FBS and gently resuspending the cells by pipetting in FACS buffer (1% FBS/PBS) at 1×10 cells/mL. Anti-GPC3 antibodies diluted to an appropriate concentration was added and reacted on ice for 30 minutes. A 3-fold serial dilution (8 dilutions in total) of the purified antibody starting at a concentration of 100 nM. The cells were washed once with FACS buffer and a Goat anti-human IgG Fc-Alexa 488 (Jackson Immunochemicals) for detection were added on ice for 30 minutes. After the incubation, the cells were centrifuged at 1500 rpm for 3 minutes, and the supernatant was removed. The cells were suspended in 100 μL of FACS buffer and subjected to flow cytometry. CytoFlex (Beckman Coulter) was used as a flow cytometer.

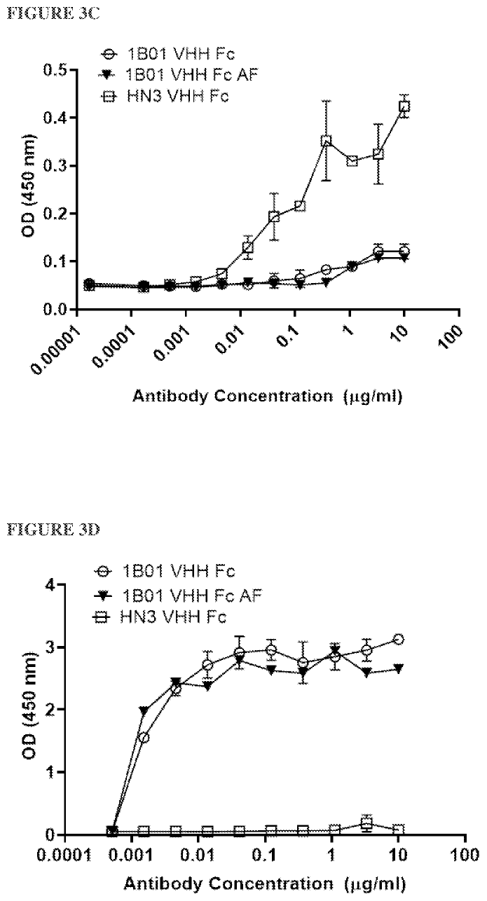

As shown in FIGS. 4A and 4B, anti-GPC3 antibodies 1B01 VHH-Fc and HN3 VHH-Fc both bound to HepG2 and Hep3B hepatoma cell lines in dose-dependent manners. 1B01 showed stronger binding to HepG2 cells, whereas binding of 1B01 and HN3 to HepG2 cells was similar. This difference was likely due to avidity, as HepG2 cells express a higher level of GPC3 compared to Hep3B cells.

Furthermore, the ability of 1B01 to elicit antibody-dependent cell-mediated cytotoxicity (ADCC) against human hepatoma cell lines was evaluated. Briefly, human peripheral blood mononuclear cells (hPBMCs) were isolated from heparinized blood samples by gradient centrifugation. Target cells, the HepG2 and Hep3B cells, were labeled with BATDA bis(acetoxymethyl) 2,2′:6′,2″-terpyridine-6,6″-dicarboxylate) DELFIA reagent (the Dissociation-Enhanced Lanthanide Fluorescent Immunoassay, Perkin Elmer) at a density of 1×10⁶ cells per ml for 30 min at 37° C., then washed with growth media and seeded at a cell density of 5×10 3 cells per well. Target cells were co-cultured with hPBMCs from four healthy donor at an effector-to-target (ET) ratio of 40-fold. After two hours incubation at 37° C., target cell lysis was measured via time-resolved fluorescence (TRF) using Varioskan LUX (Thermo Fisher Scientific). An anti-CD20 antibody was used a negative control.

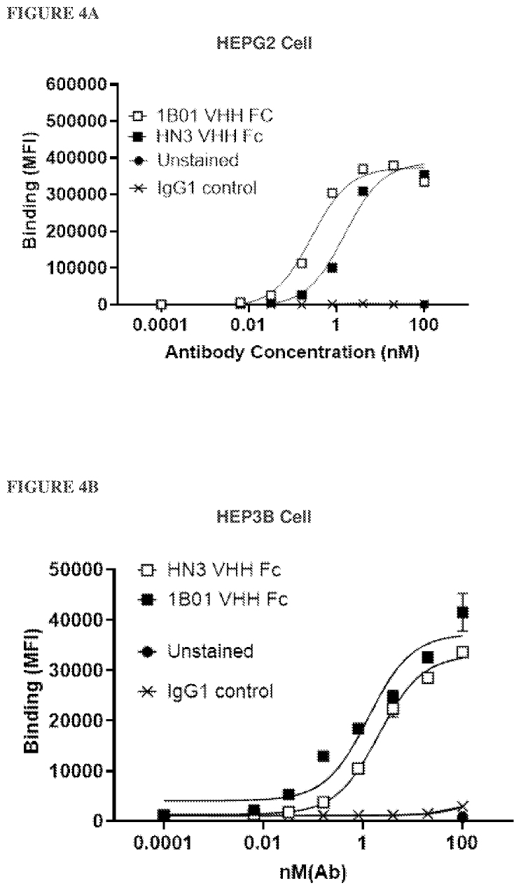

As shown in FIG. 5, 1B01 VHH-Fc, its afucosylated version (AF) and HN3 VHH-Fc all showed dose-dependent tumor lysis compared to the control, and both versions of 1B01 showed stronger tumor lysis at lower concentrations compared to the HN3 analog.

### Example 4. In Vivo Antitumor Efficacy of Anti-GPC3 Antibody 1B01

A xenograft mouse model was used to determine the in vivo antitumor efficacy of 1B01. HepG2 cells were prepared at 5×10⁶ cells/mL in a solution containing 100 μl PBS medium and MATRIGEL (BD Bioscience) at a ratio of 1:1, and subcutaneously implanted into both side flank of BALB/c nude mice (Biolasco, Taipei, Taiwan). Tumors were observed and measured twice a week until the end of the study. Tumor volume was defined as TV=0.5 a×b², where a is the long diameter of the tumor and b is the short diameter of the tumor. Treatment began on day 7-8 when the average tumor volume reached 150 mm³ (n=8 mice per group). The antibodies and vehicle (saline solution) were administered intraperitoneally twice a week over six doses.

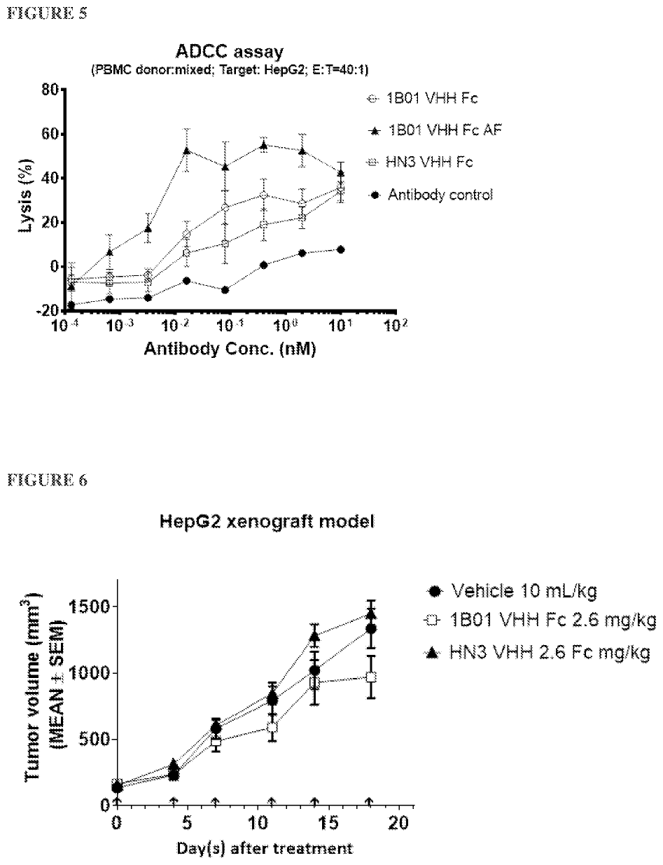

As shown in FIG. 6, at a relatively low dose of 2.6 mg/kg, treatment using 1B01 led to reduced tumor volume compared to the vehicle control group. The tumor growth inhibition (TGI) rate was about 30%. In contrast, treatment using HN3 at the same dose did not show reduced tumor volume compared to the vehicle control group. Although both 1B01 and HN3 can inhibit tumor growth at much higher doses (data not shown), the results at the low dose indicate that 1B01 can have enhanced antitumor efficacy compared to the HN3 analog in solid tumors where therapeutic antibodies have difficulty to reach a high concentration in the tumor microenvironment.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents.

Various publications, patents and patent applications are cited herein, the contents of which are hereby incorporated by reference in their entireties.

1-70. (canceled)

71. An antibody that binds to GPC3, comprising a single domain antibody that binds to GPC3, wherein the single domain antibody comprises:
a) a heavy chain variable region CDR1 comprising the amino acid sequence set forth in SEQ ID NO: 1, a heavy chain variable region CDR2 comprising the amino acid sequence set forth in SEQ ID NO: 2, and a heavy chain variable region CDR3 comprising the amino acid sequence set forth in SEQ ID NO: 3; or
b) a heavy chain variable region CDR1 comprising the amino acid sequence set forth in SEQ ID NO: 5, a heavy chain variable region CDR2 comprising the amino acid sequence set forth in SEQ ID NO: 6, and a heavy chain variable region CDR3 comprising the amino acid sequence set forth in SEQ ID NO: 7.

72. The antibody of claim 71, wherein the single domain antibody comprises a heavy chain variable region CDR1 comprising the amino acid sequence set forth in SEQ ID NO: 1, a heavy chain variable region CDR2 comprising the amino acid sequence set forth in SEQ ID NO: 2, and a heavy chain variable region CDR3 comprising the amino acid sequence set forth in SEQ ID NO: 3.

73. The antibody of claim 71, wherein the single domain antibody comprises a heavy chain variable region CDR1 comprising the amino acid sequence set forth in SEQ ID NO: 5, a heavy chain variable region CDR2 comprising the amino acid sequence set forth in SEQ ID NO: 6, and a heavy chain variable region CDR3 comprising the amino acid sequence set forth in SEQ ID NO: 7.

74. The antibody of claim 71, wherein the single domain antibody comprises a heavy chain variable region comprising the amino acid sequence set forth in SEQ ID NO: 4.

75. The antibody of claim 71, wherein the single domain antibody comprises a heavy chain variable region comprising the amino acid sequence set forth in SEQ ID NO: 8.

76. The antibody of claim 71, wherein the single domain antibody comprises a heavy chain variable region comprising the amino acid sequence set forth in SEQ ID NO: 12.

77. The antibody of claim 71, wherein the antibody comprises a Fc region.

78. The antibody of claim 77, wherein the Fc region comprises a Fc region selected from the group consisting of the Fc regions of IgG1, IgG2, IgG3 and IgG4.

79. The antibody of claim 78, wherein the IgG1 Fc region comprises one or more mutation that enhances an antibody-dependent cell-mediated cytotoxicity (ADCC), wherein the IgG1 Fc region comprises the mutations of L235V, F243L, R292P and Y300L, the mutations of S239D, A330L and I332E, or the mutations of L235V, F243L, R292P, Y300L and P396L.

80. An immunoconjugate comprising the antibody of claim 71, linked to a therapeutic agent or a label.

81. A chimeric antigen receptor (CAR) comprising an extracellular antigen-binding domain that comprises an antibody of claim 71.

82. An immunoresponsive cell comprising a CAR of claim 81.

83. A pharmaceutical composition comprising a) the antibody of claim 71, and b) a pharmaceutically acceptable carrier.

84. A nucleic acid encoding the antibody of claim 71.

85. A vector comprising the nucleic acid of claim 84.

86. A host cell comprising the nucleic acid of claim 84.

87. A method for preparing an antibody of claim 71 comprising expressing the antibody in a host cell comprising a nucleic acid encoding the antibody of claim 71 and isolating the antibody from the host cell.

88. A method of reducing tumor burden in a subject, the method comprising administering to the subject an effective amount of a pharmaceutical composition of claim 83.

89. A kit comprising an antibody of claim 71.


SEQUENCE LISTING (50 sequences)

SEQ ID NO: 1 - protein, 8 aa, synthetic construct
  DOMAIN 1..8: note = 4F3 llama CDR1
GFTFSSYI

SEQ ID NO: 2 - protein, 8 aa, synthetic construct
  DOMAIN 1..8: note = 4F3 llama CDR2
ISTGGKST

SEQ ID NO: 3 - protein, 12 aa, synthetic construct
  DOMAIN 1..12: note = 4F3 llama CDR3
AKGGKSRSYYSE

SEQ ID NO: 4 - protein, 119 aa, synthetic construct
  DOMAIN 1..119: note = 4F3 llama VHH
QVQLQESGGGLVQPGGSLRLSCAASGFTFSSYIMSWIRQAPGKELEWVATISTGGKSTAYADSVKGRFTVSRDNAINTAYLQMNSLKSEDTAVYYCAKGGKSRSYYSERGQGTLVTVSS

SEQ ID NO: 5 - protein, 8 aa, synthetic construct
  DOMAIN 1..8: note = 1B01 llama CDR1
GLPFSNYA

SEQ ID NO: 6 - protein, 8 aa, synthetic construct
  DOMAIN 1..8: note = 1B01 llama CDR2
VSANGGNE

SEQ ID NO: 7 - protein, 15 aa, synthetic construct
  DOMAIN 1..15: note = 1B01 llama CDR3
ATVRRRGGTFTVGSY

SEQ ID NO: 8 - protein, 122 aa, synthetic construct
  DOMAIN 1..122: note = 1B01 llama VHH
QVQLQESGGGLVQAGGSLRLSCAAVGLPFSNYAMGWFRQAPGEEREFVSAVSANGGNEYYADSVKDRFTISRDNAKNTVYLRMLSLKLEDTAIYYCATVRRRGGTFTVGSYRGQGTQVTVSS

SEQ ID NO: 9 - protein, 8 aa, synthetic construct
  DOMAIN 1..8: note = 1B01 CDR1
GLPFSNYA

SEQ ID NO: 10 - protein, 8 aa, synthetic construct
  DOMAIN 1..8: note = 1B01 CDR2
VSANGGNE

SEQ ID NO: 11 - protein, 15 aa, synthetic construct
  DOMAIN 1..15: note = 1B01 CDR3
ATVRRRGGTFTVGSY

SEQ ID NO: 12 - protein, 122 aa, synthetic construct
  DOMAIN 1..122: note = 1B01 VHH
QVQLVESGGGLVQPGGSLRLSCAAVGLPFSNYAMGWFRQAPGKGLEFVSAVSANGGNEYYADSVKGRFTISRDNSKNTLYLQMNSLRAEDTAVYYCATVRRRGGTFTVGSYRGQGTQVTVSS

SEQ ID NO: 13 - protein, 354 aa, synthetic construct
  DOMAIN 1..354: note = 1B01 VHH Fc
QVQLVESGGGLVQPGGSLRLSCAAVGLPFSNYAMGWFRQAPGKGLEFVSAVSANGGNEYYADSVKGRFTISRDNSKNTLYLQMNSLRAEDTAVYYCATVRRRGGTFTVGSYRGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 603 aa, synthetic construct
  PEPTIDE 1..603: note = Human GPC3 polypeptide
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTETEKKIWHFKYPIFFLCIGLDLQIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 15 - protein, 196 aa, synthetic construct
  DOMAIN 1..196: note = C-terminal ECD of Human GPC3 polypeptide
SAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHN

SEQ ID NO: 16 - protein, 10 aa, synthetic construct
  DOMAIN 1..10: note = Exemplary linker16
GGGGSGGGGS

SEQ ID NO: 17 - protein, 12 aa, synthetic construct
  DOMAIN 1..12: note = Exemplary linker17
GSGGSGGSGGSG

SEQ ID NO: 18 - protein, 15 aa, synthetic construct
  DOMAIN 1..15: note = Exemplary linker18
GGGGSGGGGSGGGGS

SEQ ID NO: 19 - protein, 5 aa, synthetic construct
  DOMAIN 1..5: note = Exemplary linker19
GGGSG

SEQ ID NO: 20 - protein, 10 aa, synthetic construct
  DOMAIN 1..10: note = Exemplary linker20
GGGSGGGGSG

SEQ ID NO: 21 - protein, 8 aa, synthetic construct
  DOMAIN 1..8: note = Exemplary linker21
GGSGGGSG

SEQ ID NO: 22 - protein, 12 aa, synthetic construct
  DOMAIN 1..12: note = Exemplary linker22
GGSGGGSGGGSG

SEQ ID NO: 23 - protein, 6 aa, synthetic construct
  DOMAIN 1..6: note = Exemplary linker23
GSGGSG

SEQ ID NO: 24 - protein, 9 aa, synthetic construct
  DOMAIN 1..9: note = Exemplary linker24
GSGGSGGSG

SEQ ID NO: 25 - protein, 7 aa, synthetic construct
  DOMAIN 1..7: note = Exemplary linker25
GSGSGSG

SEQ ID NO: 26 - protein, 12 aa, synthetic construct
  DOMAIN 1..12: note = Exemplary linker26
GSGGSGGSGGSG

SEQ ID NO: 27 - protein, 18 aa, synthetic construct
  DOMAIN 1..18: note = Exemplary linker27
GGGGSGGGGSGGGGSGGG

SEQ ID NO: 28 - protein, 25 aa, synthetic construct
  DOMAIN 1..25: note = Exemplary linker28
GGGGSGGGGSGGGGSGGGGSGGGGS

SEQ ID NO: 29 - protein, 30 aa, synthetic construct
  DOMAIN 1..30: note = Exemplary linker29
GGGGSGGGGSGGGGSGGGGSGGGGSGGGGS

SEQ ID NO: 30 - protein, 20 aa, synthetic construct
  DOMAIN 1..20: note = Exemplary linker30
GGGGSGGGGSGGGGSGGGGS

SEQ ID NO: 31 - protein, 5 aa, synthetic construct
  DOMAIN 1..5: note = Exemplary linker31
PAPAP

SEQ ID NO: 32 - protein, 15 aa, synthetic construct
  DOMAIN 1..15: note = Exemplary linker32
PAPAPPAPAPPAPAP

SEQ ID NO: 33 - protein, 7 aa, synthetic construct
  DOMAIN 1..7: note = Exemplary linker33
IKRTVAA

SEQ ID NO: 34 - protein, 7 aa, synthetic construct
  DOMAIN 1..7: note = Exemplary linker34
VSSASTK

SEQ ID NO: 35 - protein, 10 aa, synthetic construct
  DOMAIN 1..10: note = Exemplary linker35
GGGGSGASTK

SEQ ID NO: 36 - protein, 10 aa, synthetic construct
  DOMAIN 1..10: note = Exemplary linker36
ASTKGGGGSG

SEQ ID NO: 37 - protein, 4 aa, synthetic construct
  DOMAIN 1..4: note = Exemplary linker37
ASTK

SEQ ID NO: 38 - protein, 12 aa, synthetic construct
  DOMAIN 1..12: note = Exemplary linker38
ASTKSGGSGGSG

SEQ ID NO: 39 - protein, 7 aa, synthetic construct
  DOMAIN 1..7: note = Exemplary linker39
AEAAAKA

SEQ ID NO: 40 - protein, 12 aa, synthetic construct
  DOMAIN 1..12: note = Exemplary linker40
AEAAAKEAAAKA

SEQ ID NO: 41 - protein, 10 aa, synthetic construct
  DOMAIN 1..10: note = Exemplary linker41
GRPGSGRPGS

SEQ ID NO: 42 - protein, 20 aa, synthetic construct
  DOMAIN 1..20: note = Exemplary linker42
GRPGSGRPGSGRPGSGRPGS

SEQ ID NO: 43 - protein, 10 aa, synthetic construct
  DOMAIN 1..10: note = Exemplary linker43
GRGGSGRGGS

SEQ ID NO: 44 - protein, 20 aa, synthetic construct
  DOMAIN 1..20: note = Exemplary linker44
GRGGSGRGGSGRGGSGRGGS

SEQ ID NO: 45 - protein, 10 aa, synthetic construct
  DOMAIN 1..10: note = Exemplary linker45
GKPGSGKPGS

SEQ ID NO: 46 - protein, 20 aa, synthetic construct
  DOMAIN 1..20: note = Exemplary linker46
GKPGSGKPGSGKPGSGKPGS

SEQ ID NO: 47 - protein, 10 aa, synthetic construct
  DOMAIN 1..10: note = Exemplary linker47
GEPGSGEPGS

SEQ ID NO: 48 - protein, 20 aa, synthetic construct
  DOMAIN 1..20: note = Exemplary linker48
GEGGSGEGGSGEGGSGEGGS

SEQ ID NO: 49 - protein, 10 aa, synthetic construct
  DOMAIN 1..10: note = Exemplary linker49
GDPGSGDPGS

SEQ ID NO: 50 - protein, 20 aa, synthetic construct
  DOMAIN 1..20: note = Exemplary linker50
GDPGSGDPGSGDPGSGDPGS